SEQUENCE LISTING (14 sequences)

SEQ ID NO: 1 - protein, 1252 aa, unidentified
  source 1..1252: note = Viral; note = RsYN04
MFILLLLPIVLAQQDSCNHIVQLPNSMVRGVYNSGSKVYYPDDINRVSTSILFNDYFLPFNSNLTNRQTLGNTNNNRFDVFTEPFLDGVYFAATEHSNVLRGWIFGTSFTNTTQSALFYNNGTNVVIEVCNFNFCLVPRVHALPNGSSISYIYTSKANCTYTYVSQSFSLNGAICSSNCDKFKDFREFIFKYSEGFLHVYSGYKAISQGNGFVPSFSALKPFGKFPIGILVTNFSVILTSFYHDSTTTKLAASSLFYGHLRPITMLLQYDENGTIYNAIDCSQDPLSELKCSLKAVNVSKGIYQTSNFRVTPSQSVARYPNITNLCPFSQVFNATRFPSVYAWTRERISNCIADYSVLYNSTSFSTFRCYGVSPTKLNDLCFSNVYADSMVVRGDEVRQIAPSQTGVIADYNYKLPDDFTGCVIAWNSKAKDENGQYFYRLFRKSKLLPFQRDVSNVTYGSGKNDGCNPSEADCYWPLLKYGFTSSVSQDYQPYRVVVLSFELLNAPATVCGPKRSTELVYNKCVNFNFNGLTGVGVLTNSSKKFLPFQQYGRDNSDFVNAVRDPPTNAILDITPCSFGGVSVITPGTNLTNEVAVLYQDVNCTDVPTLLSLNDYNVGWRVYSTGTSRFQIQAGCLIGAQYQNVTKECDIPIGAGVCASYTTKARTSSTPALFAYTMSLGATQSPAYANNTVLIPTNFSVSVITEVLPVSMAKTAIDCNMYICGDSTECSRLLLQYGSFCEQLNRALAGVALEQDKNTQDVFAQVKAVYKTPTFTDFGGFNFSSVLPQPGKPSQRSFIEDLLFNKVTLSDAGFMKEYGNCLGDVAARDLICAQKFNGLTVLPPLLTDEMIAQYTSSLVGGTIASGWTAGAGAALQIPFAMQMAYRFNGIGVTQNVLYENQKLIANQFNNAITQIQSSLTTTSTALSKLQDVINQNANALNTLVKQLSSNFGAISSVLNDILSRLDKVEAEVQIDRLITGRLQSAQTYVTQQLIKAAEVRASANLAATKMSECVLGQSKRVDFCGKGFHIMSFPQAAPHGVVFLHMNYIPSSERNFTTVPAICHEGRAYFPREGVFINDNGNWSITPRNYYAPSPITTENTFMSGSCDVVIGIINNTVYDPLQPELDSFKEELDKFFKNHTSPDLDLEDVSKLNASVVDINKEIEKLNNIAQQLNSSLIDLQELGKYEQYLKWPWYIWLGFIAGLIAIIMASIMLCCMTSCCSCFKGLCSCGLCCKFDDDSEPVLQGVKLHYT

SEQ ID NO: 2 - protein, 1227 aa, unidentified
  source 1..1227: note = Bat Coronavirus_RacCS271; note = Bat
MFILLLIGYTAATTCVTGPSTENKQNVSSLMRGVYYPDDIYRSNVNVLFTVPFLRFNSTLTWYNFWNQAYTSRVMAFGDGIYFSTVDKSNAVRGWIFGTTLDNTTQSALLINNGSLVTIQVCYFQFCANPIAAIVFTXXXXXXXXXSSYNCTYYEVLSHIXXXXTEDTGSFKRLREFVFKNYDGFLHIYGAYEPHVINIGATPSLPSTFKPLIPXXXLXXSLNITNFKVVLTTXXNSQPXXXXFAVGSLKLTTIMLSFDINGTIDKAVDCSSDPLSELKCTLKSFNVSKGIYPTSNFXXXXXXXXXXXXXXXXXXXXXXXXXXXXXXXXXXXXXXXXXXXXXXXXXXXXWYSCSTFKCYGVSPSKLIDLCFTSVYADTFLIRFSEVRQIAPGETGVIADYNYKLPDDFTGCVLAWNTAKQDIGSYFYXXXXXXXXXXXXXXXXXXXXXXXXXXXYDFNPNVPLDYQATRVVVLSFELLXXXXXXXXXKLSTQLVKNRCVNXXXXXXXXXGVLTDSDKRFQSFQQFGRDSADFXXXXXXXXXLXXXXXXXCSFGGVSVITPGTQTSSKVAVLYQDVNCTDVPTALGLDQISAAWRVYAIGNDVFQTQAGCLVGAEHANTSHECDIPXXXXVCASYNSPVARVGTNSIIAYEMSIGAESSIAYANNSIAIPTNFSXXXTXXXLPVSMSKTSVDCTMYICGDSQECNKLLLQYGSFCAQLNRALSGVAVEQDKNTEAVFAQVKQIYKAPVIKDLGGFNFSQLLPDPTKPSQRSFIEDLLFDKVTLSDAGFIKQYGDCLGDIAARDLICAQKFNGLTVLPPLLTDEMIAAYTSALVSGTATAGWTFGIGAALQVPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICYEGKAHFPREGVFVSNGTHWFVTQRNFYEPQIITTDNTFVSGSCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIIMVTIMLCCMTSCCSCLKGCCSCGSCCKFDEDDSEPLLKGVKLHYT

SEQ ID NO: 3 - protein, 1235 aa, unidentified
  source 1..1235: note = Japan_Rc-o319; note = Bat
MFILVLLNVLSVYGCVNITYGSHHLYVSSRTRGVYYPDDAFRSSTNVLHEGFFLPFDSNVTWYSFWNQKYSVATSPFGDGVYFSTIDKSNVVRGWVFGTTLDNDTQSVLLYNDGTHVRVEVCTFHFCPTPVFSASSPHLYSSAFNCTLNYTLASVRADFTEVDGSFKTIREFVFKLQDGSLNVYYASTSYVLAIGATSQLPSGVTPLVPLWKIPIGLNITNFKTLVYLRSDNTPLQAAYVVGHLKRRTMMFKYDENGTIVDAIDCALDPLSETKCTLRSFIVEKGIYQTSNFRVQPQDTVVRFPNITNLCPFSEVFNATTFASVYAWNRKRISNCVADYSVLYNSTSFSTFQCYGVSSTKLNDLCFTNVYADSFVVRGDEVRQIAPGQTGVIADYNYKLPDDFTGCVLAWNSRNQDASTSGNFNYYYRIWRSEKLRPFERDIAHYDYQVGTQFKSSLKNYGFYSSAGDSHQPYRVVVLSFELLNAPATVCGPKQSTELIKNKCVNFNFNGLTGTGVLTDSNKKFQSFQQFGRDVSDFTDSVKDPKTLEVLDITPCSYGGVSVITPGTNASTQVAVLYQDVNCTDVPTAIHAEQLTPSWRVYSTGTNMFQTQAGCLIGAEHVNNSYDCDIPIGAGICATYHTPSMLRSANNNKRIVAYVMSLGAENSVAYSNNTIAIPTNFTISVTTEVMPVSMTKTSVDCTMYICGDSVECSTLLLQYGSFCTQLNRALTGIAVEQDKNTQEVFAQVKQIYKTPDIKDFGGFNFSQILPDPSKPSKRSFIEDLLFNKVTLADAGFVKQYGDCLGDIQARDLICAQKFNGLTVLPPLLTDEMIAAYTAALISGTATAGWTFGAGAALQIPFAMQMAYRFNGIGVTQNVLYENQKLIANQFNSAIGKIQESLTSTASALGKLQDVVNQNAQALNTLVKQLSSNFGAISSVLNDIISRLDKVEAEVQIDRLITGRLQSLQTYVTQQLIRAAEIRASANLAATKMSECVLGQSKRVDFCGKGYHLMSFPQAAPHGVVFLHVTYIPSQERNFTTAPAICHEGKAHFPREGVFVSNGTHWFITQRNFYEPQIITTDNTFVSGTCDVVIGIVNNTVYDPLQPELESFKDELDKYFKNHTSPDIDLGDISGINASVVDIQKEIDILKDVAKNLNESLINLQELGKYEQYIKWPWYVWLGFIAGLIAIIMVTIMLCCMTSCCSCLKGCCSCGSCCKFDEDDSEPVLKGVKLHYT

SEQ ID NO: 4 - protein, 1246 aa, unidentified
  source 1..1246: note = Zhejiang_SL_CoVZC45; note = Bat
MLFFLFLQFALVNSQCVNLTGRTPLNPNYTNSSQRGVYYPDTIYRSDTLVLSQGYFLPFYSNVSWYYSLTTNNAATKRTDNPILDFKDGIYFAATEHSNIIRGWIFGTTLDNTSQSLLIVNNATNVIIKVCNFDFCYDPYLSGYYHNNKTWSIREFAVYSSYANCTFEYVSKSFMLNISGNGGLFNTLREFVFRNVDGHFKIYSKFTPVNLNRGLPTGLSVLQPLVELPVSINITKFRTLLTIHRGDPMPNNGWTAFSAAYFVGYLKPRTFMLKYNENGTITDAVDCALDPLSETKCTLKSLTVQKGIYQTSNFRVQPTQSVVRFPNITNVCPFHKVFNATRFPSVYAWERTKISDCIADYTVFYNSTSFSTFKCYGVSPSKLIDLCFTSVYADTFLIRFSEVRQVAPGQTGVIADYNYKLPDDFTGCVIAWNTAKQDVGNYFYRSHRSTKLKPFERDLSSDENGVRTLSTYDFNPNVPLEYQATRVVVLSFELLNAPATVCGPKLSTQLVKNQCVNFNFNGLKGTGVLTDSSKRFQSFQQFGKDASDFIDSVRDPQTLEILDITPCSFGGVSVITPGTNTSLEVAVLYQDVNCTDVPTTIHADQLTPAWRIYATGTNVFQTQAGCLIGAEHVNASYECDIPIGAGICASYHTASILRSTSQKAIVAYTMSLGAENSIAYANNSIAIPTNFSISVTTEVMPVSMAKTSVDCTMYICGDSIECSNLLLQYGSFCTQLNRALSGIAIEQDKNTQEVFAQVKQIYKTPPIKDFGGFNFSQILPDPSKPSKRSFIEDLLFNKVTLADAGFIKQYGDCLGGISARDLICAQKFNGLTVLPPLLTDEMIAAYTAALISGTATAGWTFGAGAALQIPFAMQMAYRFNGIGVTQNVLYENQKLIANQFNSAIGKIQESLTSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYIPSQEKNFTTAPAICHEGKAHFPREGVFVSNGTHWFVTQRNFYEPKIITTDNTFVSGNCDVVIGIINNTVYDPLQPELDSFKEELDKYFKNHTSPDIDLGDISGINASVVNIQKEIDRLNEVARNLNESLIDLQELGKYEQYIKWPWYVWLGFIAGLIAIVMVTILLCCMTSCCSCLKGCCSCGSCCKFDEDDSEPVLKGVKLHYT

SEQ ID NO: 5 - protein, 1265 aa, unidentified
  source 1..1265: note = Pangolin_coronavirus_PCoV_GD-MP789 (Pangolin_Guangdong_MP789); note = Pangolin
MLFFFFLHFALVNSQCVNLTGRAAIQPSFTNSSQRGVYYPDTIFRSNTLVLSQGYFLPFYSNVSWYYALTKTNSAEKRVDNPVLDFKDGIYFAATEKSNIVRGWIFGTTLDNTSQSLLIVNNATNVIIKVCNFQFCYDPYLSGYYHNNKTWSTREFAVYSSYANCTFEYVSKSFMLDIAGKSGLFDTLREFVFRNVDGYFKIYSKYTPVNVNSNLPIGFSALEPLVEIPAGINITKFRTLLTIHRGDPMPNNGWTVFSAAYYVGYLAPRTFMLNYNENGTITDAVDCALDPLSEAKCTLKSLTVEKGIYQTSNFRVQPTESIVRFPNITNLCPFGEVFNATTFASVYAWNRKRISNCVADYSVLYNSTSFSTFKCYGVSPTKLNDLCFTNVYADSFVVRGDEVRQIAPGQTGRIADYNYKLPDDFTGCVIAWNSNNLDSKVGGNYNYLYRLFRKSNLKPFERDISTEIYQAGSTPCNGVEGFNCYFPLQSYGFHPTNGVGYQPYRVVVLSFELLKAPATVCGPKQSTNLVKNKCVNFNFNGLTGTGVLTESSKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNTYECDIPIGAGICASYQTQTNSRSVSSQAIIAYTMSLGAENSVAYANNSIAIPTNFTISVTTEILPVSMTKTSVDCTMYICGDSIECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPSQEKNFTTTPAICHEGKAHFPREGVFVSNGTHWFVTQRNFYEPQIITTDNTFVSGSCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIIMVTIMLCCMTSCCSCLKGCCSCGSCCKFDEDDSEPVLKGVKLHYT

SEQ ID NO: 6 - protein, 1267 aa, unidentified
  source 1..1267: note = Pangolin_coronavirus_PCoV_GX-P5E (Pangolin_Guangxi_P5E); note = Pangolin
MFVFLFVLPLVSSQCVNLTTRTGIPPGYTNSSTRGVYYPDKVFRSSILHLTQDLFLPFFSNVTWFNTITYQGGSKKFDNPVLPFNDGVYFASTEKSNIIRGWIFGTTLDARTQSLLIVNNATNVVIKVCEFQFCTDPFLGVYYHNNNKTWVENEFRVYSSANNCTFEYISQPFLMDLEGKQGNFKNLREFVFKNVDGYFKIYSKHTPIDLVRDLPRGFAALEPLVDLPIGINITRFQTLLALHRSYLTPGKLESGWTTGAAAYYVGYLQQRTFLLSYNQNGTITDAVDCSLDPLSETKCTLKSLTVEKGIYQTSNFRVQPTISIVRFPNITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWNSVKQDALTGDNYGYLYRLFRKSKLKPFERDISTEIYQAGSTPCNGQVGLNCYYPLERYGFHPTTGVNYQPFRVVVLSFELLNGPATVCGPKLSTTLVKDKCVNFNFNGLTGTGVLTTSKKQFLPFQQFGRDISDTTDAVRDPQTLEILDITPCSFGGVSVITPGTNTSNQVAVLYQDVNCTEVPMAIHAEQLTPAWRVYSAGANVFQTRAGCLVGAEHVNNSYECDIPVGAGICASYHSMSSFRSVNQRSIIAYTMSLGAENSVAYSNNSIAIPTNFTISVTTEILPVSMTKTSVDCTMYICGDSIECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHEGKAHFPREGVFVSNGTHWFITQRNFYEPQIITTDNTFVSGSCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIIMVTIMLCCMTSCCSCLKGCCSCGSCCKFDEDDSEPVLKGVKLHYT

SEQ ID NO: 7 - protein, 1269 aa, unidentified
  source 1..1269: note = Pangolin_coronavirus_PCoV_GX-P2V (Pangolin_Guangxi_P2V); note = Pangolin
MFVFLFVLPLVSSQCVNLTTRTGIPPGYTNSSTRGVYYPDKVFRSSILHLTQDLFLPFFSNVTWFNTIHLNYQGGFKKFDNPVLPXNDGVYFASTEKSNIIRGWIFGTTLDARTQSLLIVNNATNVVIKVCEFQFCTDPFLGVYYHNNNKTWVENEFRVYSSANNCTFEYISQPFLMDLEGKQGNFKNLREFVFKNVDGYFKIYSKHTPIDLVRDLPRGFAALEPLVDLPIGINITRFQTLLALHRSYLTPGKLESGWTTGAAAYYVGYLQQRTFLLSYNQNGTITDAVDCSLDPLSETKCTLKSLTVEKGIYQTSNFRVQPTISIVRFPNITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWNSVKQDALTGGNYGYLYRLFRKSKLKPFERDISTEIYQAGSTPCNGQVGLNCYYPLERYGFHPTTGVNYQPFRVVVLSXELLNGPATVCGPKLSTTLVKDKCVNFNFNGLTGTGVLTTSKKQFLPFQQFGRDISDTTDAVRDPQTLEILDITPCSFGGVSVITPGTNTSNQVAVLYQDVNCTEVPMAIHAEQLTPAWRVYSAGANVFQTRAGCLVGAEHVNNSYECDIPVGAGICASYHSMSSFRSVNQRSIIAYTMSLGAENSVAYSNNSIAIPTNFTISVTTEILPVSMTKTSVDCTMYICGDSIECSNLLLQYGSFCTQLNRALTGIAVEQDKNTQEVFAQVKQIYKTPPIKDFGGFNFLQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHEGKAHFPREGVFVSNGTHWFITQRNFYEPQIITTDNTFVSGSCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIIMVTIMLCCMTSCCSCLKGCCSCGSCCKFDEDDSEPVLKGVKLHYT

SEQ ID NO: 8 - protein, 1273 aa, unidentified
  source 1..1273: note = SARs-CoV2_Spike; note = Viral
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1269 aa, unidentified
  source 1..1269: note = BatCoV_RaTG13; note = Bat
MFVFLVLLPLVSSQCVNLTTRTQLPPAYTNSSTRGVYYPDKVFRSSVLHLTQDLFLPFFSNVTWFHAIHVSGTNGIKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPPGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTDSIVRFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKSTNLVKNKCVNFNFNGLTGTGVLTESNKKFLPFQQFGRDIADTTDAVRDPQTLEILDITPCSFGGVSVITPGTNASNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGSCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIIMVTIMLCCMTSCCSCLKGCCSCGSCCKFDEDDSEPVLKGVKLHYT

SEQ ID NO: 10 - protein, 1256 aa, unidentified
  source 1..1256: note = BatCoV_Yunnan_SL_Rs7327; note = Bat
MKLLVLVFATLVSSYTIEKCLDFDDRTPPANTQFLSSHRGVYYPDDIFRSNVLHLVQDHFLPFDSNVTRFITFGLNFDNPIIPFRDGIYFAATEKSNVIRGWVFGSTMNNKSQSVIIMNNSTNLVIRACNFELCDNPFFVVLKSNNTQIPSYIFNNAFNCTFEYVSKDFNLDLGEKPGNFKDLREFVFRNKDGFLHVYSGYQPISAASGLPTGFNALKPIFKLPLGINITNFRTLLTAFPPRPDYWGTSAAAYFVGYLKPTTFMLKYDENGTITDAVDCSQNPLAELKCSVKSFEIDKGIYQTSNFRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFMGCVLAWNTRNIDATSTGNYNYKYRSLRHGKLRPFERDISNVPFSPDGKPCTPPAFNCYWPLNDYGFFTTNGIGYQPYRVVVLSFELLNAPATVCGPKLSTDLIKNQCVNFNFNGLTGTGVLTSSSKRFQPFQQFGRDVSDFTDSVRDPKTSEILDISPCSFGGVSVITPGTNTSSEVAVLYQDVNCTDVPVAIHADQLTPAWRIYSTGNNVFQTQAGCLIGAEHVDTSYECDIPIGAGICASYHTVSSLRSTSQKSIVAYTMSLGADSSIVYSNNTIAIPTNFSISITTEVMPVSMAKTSVDCNMYICGDSTECANLLLQYGSFCTQLNRALSGIAVEQDRNTREVFAQVKQMYKTPTLKDFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 11 - protein, 1255 aa, unidentified
  source 1..1255: note = Viral; note = SARs-CoV
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 12 - protein, 1255 aa, unidentified
  source 1..1255: note = Bat; note = BatCoV_Yunnan_SL_Rs4874
MFIFLFFLTLTSGSDLESCTTFDDVQAPNYPQHSSSRRGVYYPDEIFRSDTLYLTQDLFLPFYSNVTGFHTINHRFDNPVIPFKDGVYFAATEKSNVVRGWVFGSTMNNKSQSVIIINNSTNVVIRACNFELCDNPFFAVSKPTGTQTHTMIFDNAFNCTFEYISDSFSLDVAEKSGNFKHLREFVFKNKDGFLYVYKGYQPIDVVRDLPSGFNILKPIFKLPLGINITNFRAILTAFLPAQDTWGTSAAAYFVGYLKPATFMLKYDENGTITDAVDCSQNPLAELKCSVKSFEIDKGIYQTSNFRVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNTSSEVAVLYQDVNCTDVPVAIHADQLTPSWRVYSTGNNVFQTQAGCLIGAEHVDTSYECDIPIGAGICASYHTVSSLRSTSQKSIVAYTMSLGADSSIAYSNNTIAIPTNFSISITTEVMPVSMAKTSVDCNMYICGDSTECANLLLQYGSFCTQLNRALSGIAVEQDRNTREVFAQVKQMYKTPTLKDFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGSCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 13 - protein, 1238 aa, unidentified
  source 1..1238: note = RsYN09
MFIFIITSLSIVLGRELDNCTTFDDVHSPEYLQYSSSRRGVYYPDDIFRSNVLHLTQDFFLPFNSNVTGFQSINYKFDNPIIPFRDGIYFAATEKSNVVRGWAFGSTMNKQSQSVVIINNSTNVVIRACNFELCDNPFFAVLKPTMQQIPSKIFESATNCTFEYVSQAFSLDVQENSGNFKHLREFVFKNKDGFLYVYKGYQSINAVRGLPSGFNTLKPLFKLPLGINITNFRTILTAYVPSESVWGTAAAAYFVGYLKPTTFMLSYDENGTIVDAIDCSENPLSELKCTLKNFNVTKGIYQTSNFRVSPNTEVVRFPNITNRCPFDKVFNASRFPSVYAWERTKISDCVADYTVLYNSTSFSTFKCYGVSPSKLIDLCFTSVYADTFLIRFSEVRQVAPGETGVIADYNYKLPDDFTGCVIAWNTAKQDQGYYYYRSHRKTKLKPFERDLSSDDNGVRTLSTYDFNQNVPLDYQATRVVVLSFELLNAPATVCGPKLSTQLVKNQCVNFNFNGLKGTGVLTDSSKKFQSFQQFGRDASDFTDSVRDPQTLEILDISPCSFGGVSVITPGTNASSEVAVLYQDVNCTDVPTAIHADQLTPAWRVYSTGVNVFQTQAGCLIGAEHVNASYECDIPIGAGICASYHSAATLRSVGQKSIVAYTMSLGAENSIAYANNSIAIPTNFSISVTTEVMPVSMAKTSVDCTMYICGDSQECSNLLLQYGSFCTQLNRALSGIAVEQDKNTQEVFAQVKQIYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVAGNCDVVIGIINNTVYDPLQPELDSFKEELDKYFKNHTSPDVDFGDISGINASVVNIQKEIDRLNEVAKNLNESLIDLQELGKYEQYIKWPWYVWLGFIAGLIAIVMATILLCCMTSCCSCLKGACSCGSCCKFDEDDSEPVLKGVKLHYT

SEQ ID NO: 14 - protein, 1241 aa, unidentified
  source 1..1241: note = RmYN07
MKIFLFSLLFSAALAQEGCGSLSYKPQPKLAQFSSSKRGVYYNDDIFRSDVLHLTRDYFLPFHSNLTQYFSLNVDSDRQVYFDNPTLNFGDGVYFAATEKSNVIRGWIFGSTMDNSTQSAIIVNNSTHIIIRVCNFNLCKEPMFTVSRGVHFSSWVYQSAFNCTYDRVENSFQLDTAPKTGNFKDLREYVFKNHDGFLSVYHSYTPVDIIRGIPVGFSVLKPILKLPIGINITSFKVVMTMYSQTTSNFLSESAAYYVGNLKYVTFMFQFNENGTIADAVDCSQNPLAELKCTLKNFNVSKGIYQTSNFRVSPSTEVIRFPNITNRCPFDRVFNASRFPSVYAWERTKISDCVADYTVLYNSTSFSTFKCYGVSPSKLIDLCFTSVYADTFLIRFSEVRQIAPGETGVIADYNYKLPDEFTGCVIAWNTANQDQGQYYYRSSRKTKLKPFERDLSSDENGVRTLSTYDFYPSVPLEYQATRVVVLSFELLNAPATVCGPKLSTSLIKNQCVNFNFNGLKGTGVLTDSSKKFQSFQQFGRDASDFTDSVRDPHTLQILDISPCSFGGVSVITPGTNASSEVAVLYQDVNCTDVPTAIRADQLTPAWRVYSAGVNVFQTQAGCLIGAEHVNASYECDIPIGAGICASYHTASLLRNTGQKSIVAYTMSLGAENSIAYANNSIAIPTNFSISVTTEVMPVSMAKTSVDCTMYICGDSQECSNLLLQYGSFCTQLNRALSGIAVEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHQGKAHFPREGVFVSNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLKQLGKYEQYIKWPWYVWLGFIAGLIAIVMATILLCCMTSCCSCLKGACSCGSCCKFDEDDSEPVLKGVKLHYT

(19) United States
(12) Patent Application Publication
(10) Pub. No.: US 2024/0299526 A1
(43) Pub. Date: Sep. 12, 2024

(54) COMPOSITIONS AND METHODS OF MRNA THERAPEUTICS

(71) Applicant: Nutcracker Therapeutics, Inc., Emerville, CA (US)

(72) Inventors: Samuel Deutsch, Emeryville, CA (US); Daniel Frimannsson, Emeryville, CA (US); Ole Haabeth, Emeryville, CA (US); Pei-Ken Hsu, Emeryville, CA (US)

(21) Appl. No.: 18/681,132

(22) PCT Filed: Aug. 11, 2022
(86) PCT No.: PCT/US2022/040026

§ 371 Date: Feb. 5, 2024

Related U.S. Application Data
(60) Provisional application No. 63/234,194, filed on Aug. 17, 2021.

(51) Int. Cl. A61K 39/12; A61K 39/00; A61P 31/14

(52) U.S. Cl. CPC A61K 39/12; A61P 31/14; A61K 2039/53; C12N 2770/20034

(57) ABSTRACT

The presently described and disclosed technology includes, in one example, a method, comprising: extracting a sequence of a spike protein of a first virus from a first non-human mammal that is previously exposed to an infection by the first virus; identifying a target antigen specific to the spike protein; and injecting an mRNA therapeutic comprising an mRNA encoding the target antigen into a human patient that has antibodies to a second virus.

### RELATED APPLICATIONS

The present patent application claims the priority benefit of U.S. Provisional Patent Application Ser. No. 63/234,194, filed Aug. 17, 2021, the content of which is hereby incorporated by reference in its entirety into this disclosure.

### BACKGROUND

Therapeutic nucleic acids, such as mRNA, small interfering RNA (siRNA), small activating RNA (saRNA), micro RNA (miRNA), antisense oligonucleotides, ribozymes, plasmids, and immune stimulating nucleic acids, have great promise for the prevention and treatment of diseases at the genetic level. mRNA-based vaccines have the benefit of triggering robust anti-cancer immunity without the potential danger of genome integration from DNA vaccines or the limitation of antigen selection from peptide vaccines.

### SUMMARY

To overcome the challenges of pre-existing technique, several solutions are provided in the form of examples. Specifically, provided in examples herein are mRNA-based vaccination using phylogenetically distant antigen variants of the Spike protein to generate broadly neutralizing immune responses against SARS-CoV2.

Provided in one aspect of the disclosure is a method to treat a human patient having antibodies to a first virus, the method including: extracting a sequence of a spike protein of a second virus from at least one first non-human mammal that is previously exposed to an infection by the second virus; identifying a target antigen specific to the spike protein; and injecting an mRNA therapeutic comprising an mRNA encoding the target antigen into the human patient.

In one implementation of this aspect, each of the first virus and the second virus is a type of coronavirus. In another implementation of this aspect, the first virus and the second virus are not the same. In yet another implementation of this aspect, the first virus is SARS-COV-2 virus, and the second virus is SARS-COV-1 virus. In another implementation of this aspect, the first virus is SARS-COV-1 virus, and the second virus is SARS-COV-2 virus.

In one implementation of this aspect, the at least one first non-human mammal is a bat, a pangolin, a rodent, or any combinations thereof. In one implementation of this aspect, the target antigen is a phylogenetically distant antigen variant of a spike protein of the first virus. In one implementation of this aspect, the mRNA therapeutic comprises the mRNA encapsulated in a delivery vehicle composition. In one implementation of this aspect, the sequence is selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, or SEQ ID NO:14. In one implementation of this aspect, the second virus is a betacoronavirus.

Provided in one aspect of the disclosure is a method to treat a human patient having antibodies to a first virus, the method including: extracting a sequence of a spike protein of a first virus from at least one human subject that has antibodies with respect to a second virus; identifying a target antigen specific to the spike protein; and injecting an mRNA therapeutic comprising an mRNA encoding the target antigen into the human patient.

In one implementation of this aspect, the human subject and the human patient are not the same person. In one implementation of this aspect, the human patient is (a) previously exposed to an infection by the first virus, (b) previously subjected to a vaccination regiment against the first virus, or (c) both.

In one implementation of this aspect, each of the first virus and the second virus is a type of coronavirus. In another implementation of this aspect, the first virus and the second virus are not the same. In yet another implementation of this aspect, the first virus is SARS-COV-2 virus, and the second virus is SARS-COV-1 virus.

In one implementation of this aspect, the target antigen is a viral antigen specific to the first virus. In one implementation of this aspect, the mRNA therapeutic comprises the mRNA encapsulated in a delivery vehicle composition. In one implementation of this aspect, the second virus is a betacoronavirus. In one implementation of this aspect, the sequence is selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, or SEQ ID NO:14. In one implementation of this aspect, target antigen is a phylogenetically distant antigen variant of a spike protein of the first virus. In one implementation of this aspect, the method further includes identifying an antibody sequence adapted to in vivo neutralization of a plurality of strains of the second virus.

It should be appreciated that all combinations of the foregoing aspects, concepts, and implementations and additional concepts and implementations discussed in greater detail below are contemplated as being part of the inventive subject matter disclosed herein, and may be employed in any suitable combination to achieve the benefits as described here. In particular, all combinations of claimed subject matter appearing at the end of this disclosure are contemplated as being part of the inventive subject matter disclosed herein.

### BRIEF DESCRIPTION OF THE DRAWINGS

FIG. 1 depicts a schematic view of an example of a system including a microfluidic process chip;

FIG. 2 depicts an exploded perspective view of examples of components of the system of FIG. 1;

FIG. 3 depicts a top plan view of an example of a process chip that may be incorporated into the system of FIG. 1;

FIG. 4 schematically illustrates an example of a method of manufacturing an mRNA therapeutic composition;

FIGS. 5A and 5B depict the comparison of the inhibition of the alpha spike protein variant and the inhibition of the beta spike protein variant in SARS2.

FIGS. 6A and 6B depict the inhibition of the receptor binding domain from alpha (WT) or beta spike binding to ACE2.

FIG. 7 depicts flow cytometry results of the omicron spike protein and the alpha (WT) spike protein.

FIG. 8 depicts intracellular cytokine staining results using the alpha (WT) receptor binding domain protein as stimulation.

### DETAILED DESCRIPTION

In some aspects, apparatuses and methods are disclosed herein for processing therapeutic polynucleotides. In particular, these apparatuses and methods may be closed path apparatuses and methods that are configured to minimize or eliminate manual handling during operation. The closed path apparatuses and methods may provide a nearly entirely aseptic environment, and the components may provide a sterile path for processing from initial input (e.g., template) to output (e.g., compounded therapeutic). Material inputs (e.g., nucleotides, and any chemical components) into the apparatus may be sterile; and may be input into the system without requiring virtually any manual interaction.

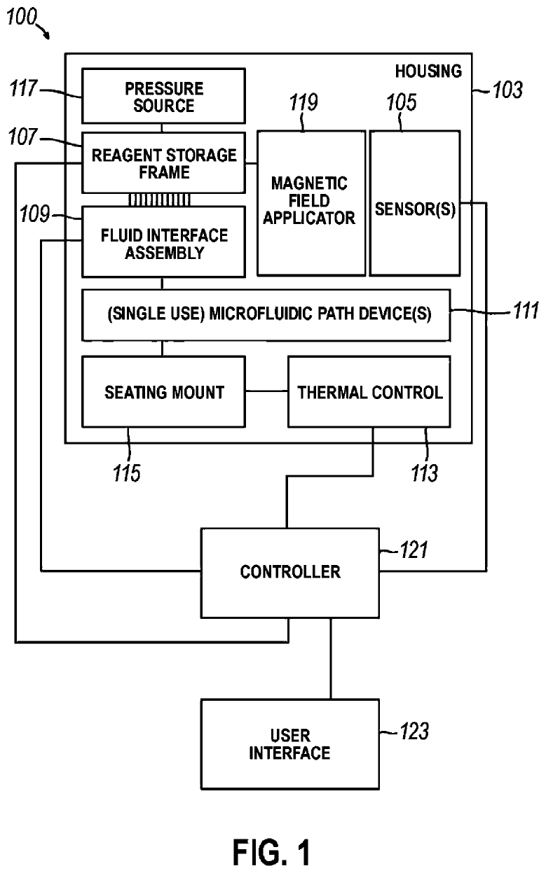

The apparatuses and methods described herein may be used to generate therapeutics at rapid cycle times at high degree of reproducibility. The apparatuses described herein may be configured to provide, in a single integrated apparatus, synthesis, purification, dialysis, compounding, and concentration of one or more therapeutic compositions. Alternatively, one or more of these processes may be carried out in two or more apparatuses as described herein. In some scenarios, the therapeutic compositions may include therapeutic polynucleotides, such as, for example, ribonucleic acids or deoxyribonucleic acids. The polynucleotides may include only natural nucleotide units or may include any kind of synthetic, semi-synthetic, or modified nucleotide units. All or some of the processing steps may be performed in an unbroken fluid processing pathway, which may be configured as one or a series of consumable microfluidic path device(s)—in some instances also referred to herein as a process chip or a biochip (though the chip need not necessarily be used in bio-related applications). The process chip in in some examples may be removably installed in an instrument that is part of a larger microfluidic system, such as that shown in FIG. 1). The disclosed apparatuses and methods may be used for the synthesis of patient-specific therapeutics, including compounding, at a point of care (e.g., hospital, clinic, pharmacy, etc.).

### Terminology

Throughout this specification and the claims which follow, unless the context requires otherwise, the word “comprise,” and variations such as “comprises” and “comprising” means various components may be co-jointly employed in the methods and articles (e.g., compositions and apparatuses including device and methods). For example, the term “comprising” will be understood to imply the inclusion of any stated elements or steps but not the exclusion of any other elements or steps. In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components, or sub-steps.

As used herein, the singular forms “a,” “an,” and “the” are intended to include the plural forms as well, unless the context clearly indicates otherwise. As used herein, the term “and/or” includes any and all combinations of one or more of the associated listed items and may be abbreviated as “/”.

Spatially relative terms, such as “under,” “below,” “lower,” “over,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features.

Thus, the term “under” may encompass both an orientation of over and under. The device may be otherwise oriented (rotated 90 degrees or at other orientations), and the spatially relative descriptors used herein interpreted accordingly. Similarly, the terms “upwardly,” “downwardly,” “vertical,” “horizontal,” and the like are used herein for the purpose of explanation only unless specifically indicated otherwise.

When a feature or element is herein referred to as being “on” another feature or element, it may be directly on the other feature or element or intervening features and/or elements may also be present. In contrast, when a feature or element is referred to as being “directly on” another feature or element, there are no intervening features or elements present. When a feature or element is referred to as being “connected,” “attached,” or “coupled” to another feature or element, it may be directly connected, attached, or coupled to the other feature or element or intervening features or elements may be present. In contrast, when a feature or element is referred to as being “directly connected,” “directly attached,” or “directly coupled” to another feature or element, there are no intervening features or elements present. Although described or shown with respect to one embodiment, the features and elements so described or shown may apply to other embodiments. It will also be appreciated by those skilled in the art that references to a structure or feature that is disposed “adjacent” another feature may have portions that overlap or underlie the adjacent feature.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is ±0.1% of the stated value (or range of values), ±1% of the stated value (or range of values), ±2% of the stated value (or range of values), ±5% of the stated value (or range of values), ±10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein.

It is also understood that when a value is disclosed as “less than or equal to” the value, “greater than or equal to the value,” and possible ranges between values are also disclosed, as appropriately understood by the skilled artisan. For example, if the value “X” is disclosed as “less than or equal to X” as well as “greater than or equal to X” (e.g., where X is a numerical value) is also disclosed. It is also understood that throughout the application, data is provided in a number of different formats and that this data represents endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

Although the terms “first” and “second” may be used herein to describe various features/elements (including steps), these features/elements should not be limited by these terms unless the context indicates otherwise. These terms are used to distinguish one feature/element from another feature/element and, unless specifically pointed out, do not denote a certain order. Thus, a first feature/element discussed below could be termed a second feature/element, and similarly, a second feature/element discussed below could be termed a first feature/element without departing from the teachings of the present invention.

As used herein, the terms “system,” “apparatus,” and “device” may be read as being interchangeable with each other. A system, apparatus, and device may each include a plurality of components having various kinds of structural and/or functional relationships with each other.

As used herein, “polynucleotide” refers to a nucleic acid molecule containing multiple nucleotides and generally refers both to “oligonucleotides” (a polynucleotide molecule of 18-25 nucleotides in length) and polynucleotides of 26 or more nucleotides. Aspects of this disclosure include compositions including oligonucleotides having a length of 18-25 nucleotides (e.g., 18-mers, 19-mers, 20-mers, 21-mers, 22-mers, 23-mers, 24-mers, or 25-mers), or medium-length polynucleotides having a length of 26 or more nucleotides (e.g., polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e.g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length. Where a polynucleotide is double-stranded, its length may be similarly described in terms of base pairs.

As used herein, “amplification” may refer to polynucleotide amplification. Amplification may include any suitable method for amplification of a polynucleotide and includes, but is not limited to, multiple displacement amplification (MDA), polymerase chain reaction (PCR) amplification, Loop Mediated Isothermal Amplification (LAMP), Nucleic Acid Sequence Based Amplification, Strand Displacement Amplification, Rolling Circle Amplification, and Ligase Chain Reaction.

As used herein, a “cassette” (e.g., a synthetic in vitro transcription facilitator cassette) refers to a polynucleotide sequence that may include or be operably linked to one or more expression elements such as an enhancer, a promoter, a leader, an intron, a 5′ untranslated region (UTR), a 3′ UTR, or a transcription termination sequence. In some aspects, a cassette comprises at least a first polynucleotide sequence capable of initiating transcription of an operably linked second polynucleotide sequence (which may comprise a template) and optionally a transcription termination sequence operably linked to the second polynucleotide sequence. The template, as described below, may comprise a sequence of interest, for example, an open reading frame (“ORF”) of interest. The cassette may be provided as a single element or as two or more unlinked elements.

As used herein, a “template” refers to a nucleic acid sequence that contains a sequence of interest for preparing a therapeutic polynucleotide according to the disclosed methods. Templates may be, but are not limited to, a double stranded DNA (dsDNA), an engineered plasmid construct, a cDNA sequence, or a linear nucleic acid sequence (for example, a linear template generated by PCR or by annealing chemically synthesized oligonucleotides). The template may, in certain aspects, be integrated into a “cassette” as described above.

As used herein, the term “sequence of interest” refers to a polynucleotide sequence, the use of which may be deemed desirable for a suitable purpose, in particular, for the manufacture of an mRNA for a therapeutic use, and includes but is not limited to, coding sequences of structural genes, and non-coding regulatory sequences that do not encode and mRNA or protein product.

As used herein, “in vitro transcription” or “IVT” refer to the process whereby transcription occurs in vitro in a non-cellular system to produce synthetic RNA molecules (e.g., synthetic mRNA) for use in various applications, including for therapeutic delivery to a subject, for example, as a therapeutic polynucleotide, which may be part of, or may be used to form, a therapeutic polynucleotide composition as described below. The therapeutic polynucleotide, (e.g., synthetic RNA molecules (transcription product)) generated may be combined with a delivery vehicle to form a therapeutic polynucleotide composition. Synthetic transcription products include mRNAs, antisense RNA molecules, shRNA, circular RNA molecules, ribozymes, and the like. An IVT reaction may use a purified linear DNA template comprising a promoter sequence and the sequence of the open reading frame (ORF) of a sequence of interest, ribonucleotide triphosphates or modified ribonucleotide triphosphates, a buffer system that includes DTT and magnesium ions, and a phage RNA polymerase.

As used herein, a “therapeutic polynucleotide” refers to a polynucleotide (e.g., an mRNA) that may be part of a therapeutic polynucleotide composition for delivery to a subject to treat a symptom, disease, or condition in a subject; prevent a symptom, disease, or condition in a subject; or to improve or otherwise modify the subject's health.

As used herein, a “therapeutic polynucleotide composition” (or “therapeutic composition” for short) may refer to a composition including one or more therapeutic polynucleotides (e.g., mRNA) encapsulated by a delivery vehicle, which composition may be administered to a subject in need thereof using any suitable administration routes, such as intratumoral, intramuscular, etc. injection. An example of a therapeutic polynucleotide composition is an mRNA (therapeutic) nanoparticle comprising at least one mRNA encapsulated by a delivery vehicle molecule. An mRNA vaccine is one example of a therapeutic polynucleotide composition.

As used herein, “delivery vehicle” refers to any substance that facilitates, at least in part, the in vivo, in vitro, or ex vivo delivery of a polynucleotide (e.g., therapeutic polynucleotide) to targeted cells or tissues (e.g., tumors, etc.). Referring to something as a delivery vehicle need not exclude the possibility of the delivery vehicle also having therapeutic effects. Some versions of a delivery vehicle may provide additional therapeutic effects. In some versions, a delivery vehicle may be a peptoid molecule, such as an amino-lipidated peptoid molecule, that may be used to at least partially encapsulate mRNA. The term “DV” will also be used herein as a shorthand for “delivery vehicle.”

As used herein, “joining” refers to methods such as ligation, synthesis, primer extension, annealing, recombination, or hybridization used to couple one component to another.

As used herein, “purifying” refers to the physical and/or chemical separation of a component (e.g., particles) of other unwanted components (e.g., contaminating substances, fragments, etc.).

As used herein, the term “substantially free” as used with respect to a given substance, includes 100% free of a given substance, or which comprises less than about 1.0%, or less than about 0.5%, or less than about 0.1% of the given substance.

As used herein, “substantially translucent” means that at least 70% (including in some instances transparency—e.g., 100%) of light is transmitted through a material.

### Overview of System Including Microfluidic Process Chip

FIG. 1 depicts examples of various components that may be incorporated into a system (100). System (100) of this example includes a housing (103) enclosing a seating mount (115) that may removably hold one or more microfluidic process chips (111). In other words, system (100) includes a chip-receiving component that is configured to removably accommodate a process chip (111), where the process chip (111) itself defines one or more microfluidic channels or fluid pathways. Components of the system (100) (e.g., within housing (103)) that fluidically interact with process chip (111) may include fluid channels or pathways that are not necessarily considered microfluidic (e.g., with such fluid channels or pathways being larger than the microfluidic channels or fluid pathways in process chip (111)). In some versions, process chips (111) are provided and utilized as single-use devices, while the rest of the system (100) is reusable. Housing (103) may be in the form of a chamber, enclosure, etc., with an opening that may be closed (e.g., via a lid or door, etc.) to thereby seal the interior. Housing (103) may enclose a thermal regulator and/or may be configured to be enclosed in a thermally-regulated environment (e.g., a refrigeration unit, etc.). Housing (103) may form an aseptic barrier. In some variations, housing (103) may form a humidified or humidity-controlled environment. In addition, or in the alternative, system (100) may be positioned in a cabinet (not shown). Such a cabinet may provide a temperature-regulated (e.g., refrigerated) environment. Such a cabinet may also provide air filtering and air flow management and may promote reagents being kept at a desired temperature through the manufacturing process. In addition, such a cabinet may be equipped with UV lamps for sterilization of process chip (111) and other components of system (100). Other suitable features may be incorporated into a cabinet that houses system (100).

In some scenarios, the assembly formed by housing (103) and the components of system (100) that are within housing (103), without process chip (111), may be considered as being an “instrument.” While controller (121) and user interface (123) are shown in FIG. 1 as being outside of housing (103), controller (121) and user interface (123) may in fact be provided in or on housing (103) and may thus also form part of the instrument. As described in greater detail below, this instrument may removably receive process chip (111) via a seating mount (115). When process chip (111) is seated in seating mount (115), the instrument and process chip (111) cooperate to together form system (100). When process chip (111) is removed from seating mount (115), the portion of system (100) that is left may be regarded as the “instrument.” The instrument, the system (100), and process chip (111) may each be considered an “apparatus.” The term “apparatus” may thus be read to include the instrument by itself, a process chip (111) by itself, the combination of the instrument and process chip (111), some other combination of components of system (100), or some other permutation of system (100) or components thereof.

Seating mount (115) may be configured to secure process chip (111) using one or more pins or other components configured to hold process chip (111) in a fixed and predefined orientation. Seating mount (115) may thus facilitate process chip (111) being held at an appropriate position and orientation in relation to other components of system (100). In the present example, seating mount (115) is configured to hold process chip (111) in a horizontal orientation, such that process chip (111) is parallel with the ground.

In some variations, a thermal control (113) may be located adjacent to seating mount (115), to modulate the temperature of any process chip (111) mounted in seating mount (115). Thermal control (113) may include a thermoelectric component (e.g., Peltier device, etc.) and/or one or more heat sinks for controlling the temperature of all or a portion of any process chip (111) mounted in seating mount (115). In some variations, more than one thermal control (113) may be included, such as to separately regulate the temperature of different ones of one or more regions of process chip (111). Thermal control (113) may include one or more thermal sensors (e.g., thermocouples, etc.) that may be used for feedback control of process chip (111) and/or thermal control (113).

As shown in FIG. 1, a fluid interface assembly (109) couples process chip (111) with a pressure source (117), thereby providing one or more paths for fluid (e.g., gas) at a positive or negative pressure to be communicated from pressure source (117) to one or more interior regions of process chip (111) as will be described in greater detail below.

While only one pressure source (117) is shown, system (100) may include two or more pressure sources (117). In some scenarios, pressure may be generated by one or more sources other than pressure source (117). For instance, one or more vials or other fluid sources within reagent storage frame (107) may be pressurized. In addition, or in the alternative, reactions and/or other processes carried out on process chip (111) may generate additional fluid pressure. In the present example, fluid interface assembly (109) also couples process chip (111) with a reagent storage frame (107), thereby providing one or more paths for liquid reagents, etc., to be communicated from reagent storage frame (107) to one or more interior regions of process chip (111) as will be described in greater detail below.

In some versions, pressurized fluid (e.g., gas) from at least one pressure source (117) reaches fluid interface assembly (109) via reagent storage frame (107), such that reagent storage frame (107) includes one or more components interposed in the fluid path between pressure source (117) and fluid interface assembly (109). In some versions, one or more pressure sources (117) are directly coupled with fluid interface assembly, such that the positively pressurized fluid (e.g., positively pressurized gas) or negatively pressurized fluid (e.g., suction or other negatively pressurized gas) bypasses reagent storage frame (107) to reach fluid interface assembly (109). Regardless of whether the fluid interface assembly (109) is interposed in the fluid path between pressure source (117) and fluid interface assembly (109), fluid interface assembly (109) may be removably coupled to the rest of system (100), such that at least a portion of fluid interface assembly (109) may be removed for sterilization between uses. As described in greater detail below, pressure source (117) may selectively pressurize one or more chamber regions on process chip (111). In addition, or in the alternative, pressure source may also selectively pressurize one or more vials or other fluid storage containers held by reagent storage frame (107).

Reagent storage frame (107) is configured to contain a plurality of fluid sample holders, each of which may hold a fluid vial that is configured to hold a reagent (e.g., nucleotides, solvent, water, etc.) for delivery to process chip (111). In some versions, one or more fluid vials or other storage containers in reagent storage frame (107) may be configured to receive a product from the interior of the process chip (111). In addition, or in the alternative, a second process chip (111) may receive a product from the interior of a first process chip (111), such that one or more fluids are transferred from one process chip (111) to another process chip (111). In some such scenarios, the first process chip (111) may perform a first dedicated function (e.g., synthesis, etc.) while the second process chip (111) performs a second dedicated function (e.g., encapsulation, etc.). Reagent storage frame (107) of the present example includes a plurality of pressure lines and/or a manifold configured to divide one or more pressure sources (117) into a plurality of pressure lines that may be applied to process chip (111). Such pressure lines may be independently or collectively (in sub-combinations) controlled.

Fluid interface assembly (109) may include a plurality of fluid lines and/or pressure lines where each such line includes a biased (e.g., spring-loaded) holder or tip that individually and independently drives each fluid and/or pressure line to process chip (111) when process chip (111) is held in seating mount (115). Any associated tubing (e.g., the fluid lines and/or the pressure lines) may be part of fluid interface assembly (109) and/or may connect to fluid interface assembly (109). In some versions, each fluid line comprises a flexible tubing that connects between reagent storage frame (107), via a connector that couples the vial to the tubing in a locking engagement (e.g., ferrule) and process chip (111). In some versions, the ends of the fluid lines/pressure lines may be configured to seal against process chip (111) (e.g., at a corresponding sealing port formed in process chip (111)), as described below. In the present example, the connections between pressure source (117) and process chip (111), and the connections between vials in reagent storage frame (107) and process chip (111), all form sealed and closed paths that are isolated when process chip (111) is seated in seating mount (115). Such sealed, closed paths may provide protection against contamination when processing therapeutic polynucleotides.

The vials of reagent storage frame (107) may be pressurized (e.g., >1 atm pressure, such as 2 atm, 3 atm, 5 atm, or higher). In some versions, the vials may be pressurized by pressure source (117). Negative or positive pressure may thus be applied. For example, the fluid vials may be pressurized to between about 1 and about 20 psig (e.g., 5 psig, 10 psig, etc.).

Alternatively, a vacuum (e.g., about −7 psig or about 7 psia) may be applied to draw fluids back into the vials (e.g., vials serving as storage depots) at the end of the process. The fluid vials may be driven at lower pressure than the pneumatic valves as described below, which may prevent or reduce leakage. In some variations, the difference in pressure between the fluid and pneumatic valves may be between about 1 psi and about 25 psi (e.g., about 3 psi, about 5 psi, 7 psi, 10 psi, 12 psi, 15 psi, 20 psi, etc.).

System (100) of the present example further includes a magnetic field applicator (119), which is configured to create a magnetic field at a region of the process chip (111). Magnetic field applicator (119) may include a movable head that is operable to move the magnetic field to thereby selectively isolate products that are adhered to magnetic capture beads within vials or other storage containers in reagent storage frame (107).

System (100) of the present example further includes one or more sensors (105). In some versions, such sensors (105) include one or more cameras and/or other kinds of optical sensors. Such sensors (105) may sense one or more of a barcode, a fluid level within a fluid vial held within reagent storage frame (107), fluidic movement within a process chip (111) that is mounted within seating mount (115), and/or other optically detectable conditions. In versions where a sensor (105) is used to sense barcodes, such barcodes may be included on vials of reagent storage frame (107), such that sensor (105) may be used to identify vials in reagent storage frame (107). In some versions, a single sensor (105) is positioned and configured to simultaneously view such barcodes on vials in reagent storage frame (107), fluid levels in vials in reagent storage frame (107), fluidic movement within a process chip (111) that is mounted within seating mount (115), and/or other optically detectable conditions. In some other versions, more than one sensor (105) is used to view such conditions. In some such versions, different sensors (105) may be positioned and configured to separately view corresponding optically detectable conditions, such that a sensor (105) may be dedicated to a particular corresponding optically detectable condition.

In versions where sensors (105) include at least one optical sensor, visual/optical markers may be used to estimate yield. For example, fluorescence may be used to detect process yield or residual material by tagging with fluorophores. In addition, or in the alternative, dynamic light scattering (DLS) may be used to measure particle size distributions within a portion of the process chip (111) (e.g., such as a mixing portion of process chip (111)). In some variations, sensor (105) may provide measurements using one or two optical fibers to convey light (e.g., laser light) into process chip (111); and detect an optical signal coming out of process chip (111). In versions where sensor (105) optically detects process yield or residual material, etc., sensor (105) may be configured to detect visible light, fluorescent light, an ultraviolet (UV) absorbance signal, an infrared (IR) absorbance signal, and/or any other suitable kind of optical feedback.

In versions where sensors (105) include at least one optical sensor that is configured to capture video images, such sensors (105) may record at least some activity on process chip (111). For example, an entire run for synthesizing and/or processing a material (e.g., a therapeutic RNA) may be recorded by one or more video sensors (105), including a video sensor (105) that may visualize process chip (111) (e.g., from above). Processing on process chip (111) may be visually tracked and this video record may be retained for later quality control and/or processing. Thus, the video record of the processing may be saved, stored, and/or transmitted for subsequent review and/or analysis. In addition, as will be described in greater detail below, the video may be used as a real-time feedback input that may affect processing using at least visually observable conditions captured in the video.

System (100) of the present example may be controlled by a controller (121). Controller (121) may include one or more processors, one or more memories, and various other suitable electrical components. In some versions, one or more components of controller (121) (e.g., one or more processors, etc.) is/are embedded within system (100) (e.g., contained within housing (103)). In addition, or in the alternative, one or more components of controller (121) (e.g., one or more processors, etc.) may be detachably attached or detachably connected with other components of system (100). Thus, at least a portion of controller (121) may be removable. Moreover, at least a portion of controller (121) may be remote from housing (103) in some versions.

The control by controller (121) may include activating pressure source (117) to apply pressure through process chip (111) to drive fluidic movement, among other tasks.

Controller (121) may be completely or partially outside of housing (103); or completely or partially inside of housing (103). Controller (121) may be configured to receive user inputs via a user interface (123) of system (100); and provide outputs to users via user interface (123). In some versions, controller (121) is fully automated to a point where user inputs are not needed. In some such versions, user interface (123) may provide only outputs to users. User interface (123) may include a monitor, a touchscreen, a keyboard, and/or any other suitable features.

Controller (121) may coordinate processing, including moving one or more fluid(s) onto and on process chip (111), mixing one or more fluids on process chip (111), adding one or more components to process chip (111), metering fluid in process chip (111), regulating the temperature of process chip (111), applying a magnetic field (e.g., when using magnetic beads), etc. Controller (121) may receive real-time feedback from sensors (105) and execute control algorithms in accordance with such feedback from sensors (105). Such feedback from sensors (105) may include, but need not be limited to, identification of reagents in vials in reagent storage frame (107), detected fluid levels in vials in reagent storage frame (107), detected movement of fluid in process chip (111), fluorescence of fluorophores in fluid in process chip (111), etc. Controller (121) may include software, firmware and/or hardware. Controller (121) may also communicate with a remote server, e.g., to track operation of the apparatus, to re-order materials (e.g., components such as nucleotides, process chips (111), etc.), and/or to download protocols, etc.

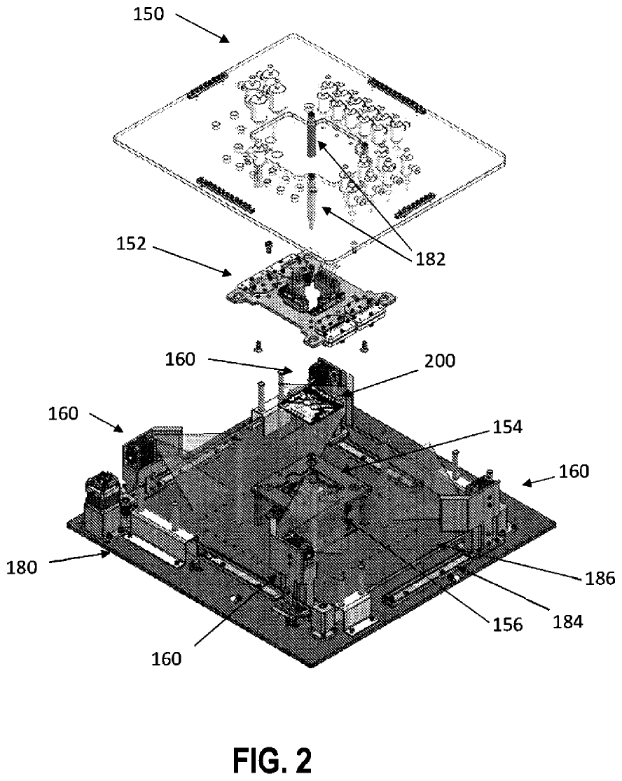

FIG. 2 shows examples of certain forms that may be taken by various components of system (100). In particular, FIG. 2 shows a reagent storage frame (150), a fluid interface assembly (152), a seating mount (154), a thermal control (156), and a process chip (200). Reagent storage frame (150), fluid interface assembly (152), seating mount (154), thermal control (156), and process chip (200) of this example may be configured and operable just like reagent storage frame (107), fluid interface assembly (109), seating mount (115), thermal control (113), and process chip (111), respectively, described above. These components are secured relative to a base (180). A set of rods (182) support reagent storage frame (150) over fluid interface assembly (152).

As shown in FIG. 2, a set of optical sensors (160) are positioned at four respective locations along base (180). Optical sensors (160) may be configured and operable like sensors (105) described above. Optical sensors (160) may include off-the-shelf cameras or any other suitable kinds of optical sensors. Optical sensors (160) are positioned such that fluid vials held within reagent storage frame (150) are within the field of view of one or more of optical sensors (160). In addition, process chip (200) is within the field of view of one or more of optical sensors (160). Each optical sensor (160) is movably secured to base (180) via a corresponding rail (184) (e.g., in a gantry arrangement), such that each optical sensor (160) is configured to translate laterally along each corresponding rail (184). A linear actuator (186) is secured to each optical sensor (160) and is thereby operable to drive lateral translation of each optical sensor (160) along the corresponding rail (184). Each actuator (186) may be in the form of a drive belt, a drive chain, a drive cable, or any other suitable kind of structure. Controller (121) may drive operation of actuators (186). Optical sensors (160) may be moved along rails (184) during operation of system (100) in order to facilitate viewing of the appropriate regions of vials in reagent storage frame (150) and/or process chip (200). In some scenarios, optical sensors (160) move in unison along corresponding rails (184). In some other scenarios, optical sensors (160) move independently along corresponding rails (184).

While optical sensors (160) are shown in FIG. 2 as being mounted to base (180), optical sensors (160) may be positioned elsewhere within system (100), in addition to or as an alternative to being mounted to base (180). For instance, some versions of reagent storage frame (107) may include one or more optical sensors (160) positioned and configured to provide an overhead field of view. In some such versions, such optical sensors (160) may be mounted to rails, movable cantilever arms, or other structures that allow such optical sensors (160) to be repositioned during operation of system (100).

Optical sensors (160) may be positioned in any other suitable locations. While not shown, system (100) may also include one or more sources of light (e.g., electroluminescent panels, etc.) to provide illumination that aids in optical sensing by optical sensors (160).

In some versions, one or more mirrors are used to facilitate visualization of components of system (100) by optical sensors (160). Such mirrors may allow optical sensors (160) to view components of system (100) that may not otherwise be within the field of view of sensors (160). Such mirrors may be placed directly adjacent to optical sensors (160). In addition, or in the alternative, such mirrors may be placed adjacent to one or more components of system (100) that are to be viewed by optical sensors (160).

In use of system (100), an operator may select a protocol to run (e.g., from a library of preset protocols), or the user may enter a new protocol (or modify an existing protocol), via user interface (123). From the protocol, controller (121) may instruct the operator which kind of process chip (111) to use, what the contents of vials in reagent storage frame (107) should be, and where to place the vials in reagent storage frame (107). The operator may load process chip (111) into seating mount (115); and load the desired reagent vials and export vials into reagent storage frame (107). System (100) may confirm the presence of the desired peripherals, identify process chip (111), and scan identifiers (e.g., barcodes) for each reagent and product vial in reagent storage frame (107), facilitating the vials to match the bill-of-reagents for the selected protocol. After confirming the starting materials and equipment, controller (121) may execute the protocol. During execution, valves and pumps are actuated to deliver reagents as described in greater detail below, reagents are blended, temperature is controlled, and reactions occur, measurements are made, and products are pumped to destination vials in reagent storage frame (107).

### Example of Process Chip

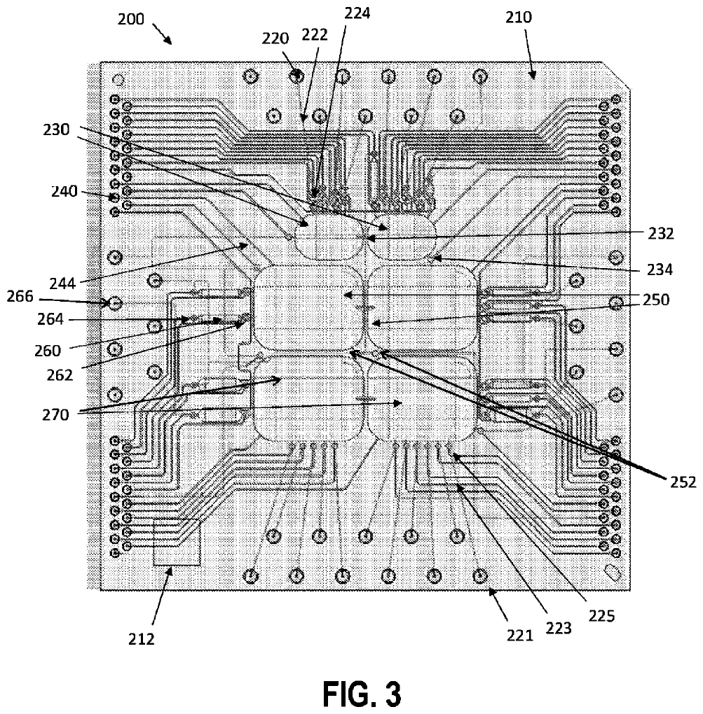

FIG. 3 depicts the example of a process chip (200) in further detail. In combination with the rest of system (100), process chip (200) may be utilized to provide in vitro synthesis, purification, concentration, formulation, and analysis of therapeutic compositions, including but not limited to therapeutic polynucleotides and therapeutic polynucleotide compositions. As shown in FIG. 3, process chip (200) of this example includes a plurality of fluid ports (220). Each fluid port (220) has an associated fluid channel (222) formed in process chip (200), such that fluid communicated into fluid port (220) will flow through the corresponding fluid channel (222). As described in greater detail below, each fluid port (220) is configured to receive fluid from a corresponding fluid line (206) from fluid interface assembly (109). In the present example, each fluid channel (222) leads to a valve chamber (224), which is operable to selectively prevent or permit fluid from the corresponding fluid channel (222) to be further communicated along process chip (200) as will be described in greater detail below.

As also shown in FIG. 3, process chip (200) of this example includes a plurality of additional chambers (230, 250, 270) that may be used to serve different purposes during the process of producing the therapeutic composition as described herein. By way of example only, such additional chambers (230, 250, 270) may be used to provide synthesis, purification, dialysis, compounding, and concentration of one or more therapeutic compositions; or to perform any other suitable function(s). Fluid may be communicated from one chamber (230) to another chamber (230) via a fluidic connector (232). In some versions, fluidic connector (232) is operable like a valve between an open and closed state (e.g., similar to valve chamber (224)). In some other versions, fluidic connector (232) remains open throughout the process of making the therapeutic composition. In the present example, chambers (230) are used to provide synthesis of polynucleotides, though chambers (230) may alternatively serve any other suitable purpose(s).

In the example shown in FIG. 3, another valve chamber (234) is interposed between one of chambers (230) and one of chambers (250), such that fluid may be selectively communicated from chamber (230) to chamber (250). Chambers (250) are provided in a pair and are coupled with each other such that process chip (200) may communicate the fluid back and forth between chambers (250). While a pair of chambers (250) are provided in the present example, any other suitable number of chambers (250) may be used, including just one chamber (250) or more than two chambers (250). Chambers (250) may be used to provide purification of the fluid and/or may serve any of the other various purposes described herein; and may have any suitable configuration. In versions where a chamber (250) is used for purification, chamber (250) may include a material that is configured to absorb selected moieties from a fluidic mixture in chamber (250). In some such versions, the material may include a cellulose material, which may selectively absorb double-stranded mRNA from a mixture. In some such versions, the cellulose material may be inserted in only one chamber (250) of a pair of chambers (250), such that upon mixing the fluid from the first chamber (250) of the pair to the second chamber (250), mRNA and/or some other component may be effectively removed from the fluidic mixture, which may then be transferred to another pair of chambers (270) further downstream for further processing or export. Alternatively, chambers (250) may be used for any other suitable purpose.

Additional valve chambers (252) are interposed between each chamber (250) and a corresponding chamber (270), such that fluid may be selectively communicated from chambers (250) to chambers (270) via valve chambers (252). Chambers (270) are also coupled with each other such that process chip (200) may communicate the fluid back and forth between chambers (270). Chambers (270) may be used to provide mixing of the fluid and/or may serve any of the other various purposes described herein; and may have any suitable configuration.

As shown in FIG. 3, chambers (270) are also coupled with additional fluid ports (221) via corresponding fluid channels (223) and valve chambers (225). Fluid ports (221), fluid channels (223), and valve chambers (225) may be configured an operable like fluid ports (220), fluid channels (222), and valve chambers (224) described above. In some versions, fluid ports (221) are used to communicate additional fluids to chambers (270). In addition, or in the alternative, fluid ports (221) may be used to communicate fluid from process chip (200) to another device. For instance, fluid from chambers (270) may be communicated via fluid ports (221) directly to another process chip (200), to one or more vials in reagent storage frame (107), or elsewhere.

Process chip (200) further includes several reservoir chambers (260). In this example, each reservoir chamber (260) is configured to receive and store fluid that is being communicated to or from a corresponding chamber (250, 270). Each reservoir chamber (260) has a corresponding inlet valve chamber (262) and outlet valve chamber (264). Each inlet valve chamber (262) is interposed between reservoir chamber (260) and the corresponding chamber (250, 270) and is thereby operable to permit or prevent the flow of fluid between reservoir chamber (260) and the corresponding chamber (250, 270). Each outlet valve chamber (264) is operable to meter the flow of fluid between reservoir chamber (260) and a corresponding fluid port (266). In some versions, each fluid port (266) is configured to communicate fluid from a corresponding vial in reagent storage frame (107) to a corresponding reservoir chamber (260). In addition, or in the alternative, each fluid port (266) may be configured to communicate fluid from a corresponding reservoir chamber (260) to a corresponding vial in reagent storage frame (107). In the present example, reservoir chambers (260) are used to provide metering of fluid communicated to and/or from process chip (200). Alternatively, reservoir chambers (260) may be utilized for any other suitable purposes, including but not limited to pressurizing fluid that is communicated to and/or from process chip (200).

As also shown in FIG. 3, process chip (200) of this example includes a plurality of pressure ports (240). Each pressure port (240) has an associated pressure channel (244) formed in process chip (200), such that pressurized gas communicated through pressure port (240) will be further communicated through the corresponding pressure channel (244). As described in greater detail below, each pressure port (240) is configured to receive pressurized gas from a corresponding pressure line (208) from fluid interface assembly (109). In the present example, each pressure channel (244) leads to a corresponding chamber (224, 225, 230, 234, 250, 252, 260, 262, 264, 270) to thereby provide valving or peristaltic pumping via such chambers (224, 225, 230, 234, 250, 252, 260, 262, 264, 270) as described in greater detail below.

Process chip (200) may also include electrical contacts, pins, pin sockets, capacitive coils, inductive coils, or other features that are configured to provide electrical communication with other components of system (100). In the example shown in FIG. 3, process chip (200) includes an electrically active region (212) includes such electrical communication features. Electrically active region (212) may further include electrical circuits and other electrical components. In some versions, electrically active region (212) may provide communication of power, data, etc. While electrically active region (212) is shown in one particular location on process chip, electrically active region (212) may alternatively be positioned at any other suitable location or locations. In some versions, electrically active region (212) is omitted.

### Example of Method of Manufacture of Therapeutics

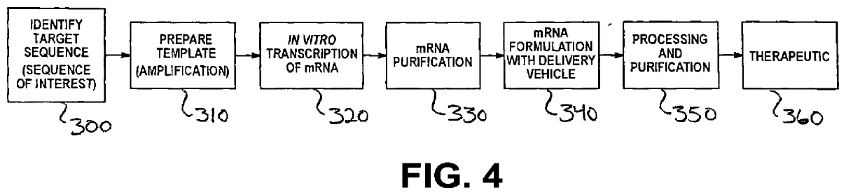

The above-described system may be used for the manufacture of mRNA-based therapeutics as described herein or other compositions. An example of a method for making an mRNA therapeutic is depicted in FIG. 4. In this example method, a target sequence (“sequence of interest”) is identified, as shown in block (300) of FIG. 4. A template comprising the target sequence (“sequence of interest”) may then be prepared and amplified (“amplification”), as shown in bock (310). Via in vitro transcription of mRNA as shown in block (320), mRNA is manufactured using a template comprising the target sequence. The resulting mRNA comprising the sequence of interest may then be purified, as shown in block (330), and then formulated with a DV, as shown in block (340). The resulting formulation comprising mRNA may then be further processed and optionally purified, as shown in block (360), for a therapeutic use, as shown in block (360). Examples of details of the method shown in FIG. 4 will be described further below.

Therapeutic uses of compositions yielded by the method shown in FIG. 4 may include, for example, cell therapies, oncological treatments, protein replacement, vaccines, expression of effector proteins, inducement of loss of function through expression of dominant negative proteins, and gene/genome editing. In addition to their high potency, mRNA therapeutics may also have benefits related to their rapid development cycle, standardized manufacturing, transient expression, and low risk of genomic integration. The methods and apparatuses described herein may be used to manufacture mRNA therapeutics for one or more of these categories of therapeutics.

### Identify Sequence of Interest

Any suitable method and criteria may be used to identify a sequence of interest for the part of the method represented by block (300) of FIG. 4. In some instances, the sequence of interest may be a short piece of DNA that encodes for some or all of a product molecule (RNA or protein). The sequence of interest may be based, at least in part, on a specific patient's genetics (e.g., genotype), including generating a specific mRNA composition based on the patient's own sequence. The sequence of interest may additionally or alternatively be based, at least in part, on a specific patient's phenotype (e.g., based on the category a patient falls into, such as risk factor categories). In any case, through the system and method described herein, a composition may be compounded at the point-of-care to generate an optimized treatment for an individual.

### Prepare Template (Amplification)

Once the sequence of interest has been identified, a template containing the sequence of interest may be prepared and amplified, as shown in block (310). The template may be a DNA template, such as linear DNA, plasmid DNA, or combinations thereof. The template may comprise an in vitro transcription facilitator cassette (IFC). The IFC may be an in vitro transcription capable double-stranded DNA. The template may be incorporated into an IFC having functional elements that facilitate in vitro transcription (e.g., from an inserted sequence of interest), such as a promoter, a portion encoding a 5′ untranslated region, (5′UTR), a portion encoding a 3′ untranslated region (3′UTR), and a portion encoding for a polyA tail. The IFC may also include one or more linkers (e.g., at least one cleavable site) useful for cloning a sequence of interest into the in vitro transcription facilitator cassette for expression of the sequence of interest and restriction sites to allow for template linearization. An IFC may be manufactured synthetically or non-synthetically.

A sequence of interest useful for inserting into an IFC may be manufactured synthetically or non-synthetically. A sequence of interest may be cleaved prior to combining it with an IFC. In particular, a sequence of interest may be cleaved with the same restriction endonuclease(s) as used to cleave the IFC; but may also be generated through enzymatic amplification. In any case, a template generated in accordance with block (310) of the method shown in FIG. 4 may take various forms. In some versions, the template comprises a uracil-containing polynucleotide sequence.

### In Vitro Transcription

A template generated in accordance with block (310) of the method shown in FIG. 4 may be used for subsequent in vitro transcription (IVT) reactions to form a therapeutic polynucleotide, such as therapeutic mRNA, as shown in block (320) of FIG. 4. This IVT process may be conducted inside a process chip such as any of the process chips (111,200) described herein, with the process being driven by controller (121).

Part of this IVT process may include combining the template with reagents such as uracil-N-glycosylase (UNG) enzyme, dNTPs (including dUTP, modified dUTP, and combinations thereof), polymerase, and buffer. The IVT reaction be incubated under controlled conditions to produce capped mRNA molecules. Following the IVT reaction, a DNAse treatment may be performed to degrade the template DNA. This may be performed inside the IVT reaction chamber, and parameters such as dilution rate, enzyme/buffer concentration, temperature and mixing may be controlled to optimized levels. This procedure may be executed autonomously and recorded by a monitoring camera (e.g., one or more of sensors (105)).

mRNA Purification

The mRNA generated through the through the IVT process may be purified, as shown in block (330), to remove impurities and side products. In some versions, this purification includes use of cellulose and an ethanol wash. For instance, a cellulose membrane may be used to selectively capture dsRNA under precisely controlled binding conditions and eluting the non-bound fraction a chamber of a process chip such as process chip (111, 200). Another purification step may use 1-2 um carboxyl-coated paramagnetic beads that selectively capture mRNA greater than 500 bp in length. One or more washes may be performed to remove unbound material, such as nucleotides, enzymes, and degraded template. Pure mRNA may then be eluted in USP grade water. A sampling chamber of a process chip (111, 200) may be used for analysis of the purified mRNA. The sampling chamber may receive detection reagents/probes for confirming the content of the resulting, purified mRNA.

mRNA Formulation with Delivery Vehicle

The purified mRNA may then be retrieved from the process chip (111, 200) for formulation with a DV, as shown in block (340). In some instances, this formulation process is carried out, at least in part, through a formulation version of process chip (111). Through the formulation process, the purified mRNA may be combined with at least one DV molecule to form an mRNA nanoparticle. For example, an aqueous solution of mRNA cargo may be combined with an ethanolic solution of DV in a microfluidic mixing structure within a formulation version of process chip (111). The material may then undergo two post-formulation processing steps involving first an on-chip dialysis process to exchange buffer components in the formulated product, followed by a concentration step to reduce the volume of the drug product to match specifications. The resulting formulation may yield encapsulated mRNA in the form of amphipathic nanoparticles (ANPs). In some versions, these ANPs are on the order of 100 nm in diameter, or smaller.

Disclosed herein, in some examples, are delivery vehicle compositions comprising hydroxyethyl-capped cationic peptoids, including, for example, hydroxyethyl-capped tertiary amino lipidated cationic peptoids. The delivery vehicle compositions of the disclosure can form an electrostatic interaction between the hydroxyethyl-capped tertiary amino lipidated cationic peptoids of the delivery vehicle composition and a polyanionic compound, such as a nucleic acid, to form a delivery vehicle complex, wherein the polyanionic compound functions as the cargo of the complex. The delivery vehicle complex is useful for the delivery of polyanionic compounds, such as nucleic acids (e.g., mRNA), into cells. Delivery vehicle complexes of the disclosure that include mRNA as the polyanionic cargo unexpectedly exhibit superior mRNA expression both in vitro and in vivo. When the mRNA of the delivery vehicle complex encodes, e.g., for a viral antigen, the delivery vehicle complexes can elicit humoral and cellular immune responses in vivo, thus functioning as a vaccine. The delivery vehicle complexes disclosed herein are further advantages in that they are stable, and demonstrate good tolerability and low toxicity.

As used herein, “peptoid” refers to a peptidomimetic compound in which one or more of the nitrogen atoms of the peptide backbone are substituted with side chains. As used herein, “lipidated peptoid” or “lipitoid” refers to a peptoid in which one or more of the side chains on the nitrogen atom comprises a lipid. As used herein, “polyanionic” refers to a compound having at least two negative charges, such as nucleic acids.

In some versions, the DV molecules may include lipitoid-based molecules, such as amino-lipidated peptoids. During the formulation process of block (340), the temperature of mixing stages on the formulation version of process chip (111) may be controlled to a temperature or range of temperatures (e.g., between about 2 degrees C. and about 20 degrees C.) that is calibrated to enhance mixing for mixing in the mixing stages. The enhanced mixing temperature may be based on the formulation being mixed (in some examples the sequence of the mRNA and/or the DV) within the particular geometry of the mixing chamber. Exposure of DV components to aqueous solution and interaction between cationic (+) lipids and anionic (−) mRNA may trigger particle formation. The mRNA may be dissolved in an acidic buffer, which may help ensure full protonation of basic functional groups (e.g., amines) on the DV responsible for its cationic charge. The DV may be dissolved in an aqueous-miscible organic solvent (e.g., ethanol) that facilitates the formation of nano-sized particles upon exposure to the aqueous cargo solution. Immediately after mixing, the solution pH may be stabilized by a neutral buffer.

In some versions, a peptoid-based lipid formulation may be used as the DV, which may incorporate both cationic groups and lipid moieties onto an N-substituted peptide (i.e., peptoid) backbone. The DV components may be monodisperse, fully-characterizable chemical entities. The DV may comprise one or more polyanionic compounds, one or more PEGylated (referring to covalent binding of polyethylene glycol (PEG) molecules) compounds, and one or more cationic compounds. Suitable cationic compounds may include but are not limited to cationic lipids, cationic lipid-peptide conjugates (e.g., lipitoids), cationic peptides, cationic polymers, and lipid-like (lipophilic) cationic compounds. The DV may comprise one or more tertiary amino lipidated and/or PEGylated cationic peptide compounds. The tertiary amino lipidated and/or PEGylated cationic peptide compounds may be peptide chains comprising N-substituted amino acid residues.

A formulation version of process chip (111) may control, with precision, the mixing rate of the material. Faster or slower mixing may be provided and controlled by controller (121). In some versions, immediately following mixing, the ANPs may be diluted with an in-line addition of 1:1 neutral PBS. This may neutralize an acidic formulation buffer and may prepare the formulation for dialysis and concentration. The ANPs created through the formulation process of block (340) may also be evaluated on the formulation version of process chip (111). For instance, the formulation process of block (340) may include a one or more dynamic light scattering (DLS) stages to evaluate particle size, particle distribution, and/or other characteristics of the ANPs. In addition, or in the alternative, a fluorescent mRNA-specific probe may be used to determine RNA concentration before and after particle disruption by addition of a detergent. This assay may elucidate the mRNA concentration for dosing information and the percentage of mRNA encapsulated in the ANPs versus free in solution. Other methods may be used.

Some description of DV complexes are provided herein. The delivery vehicle compositions disclosed herein can form complexes with one or more polyanionic compounds (e.g., nucleic acids) through an electrostatic interaction between the cationic component of the delivery vehicle composition and the polyanionic compound. The complexes, in some instances, permit a high amount of cargo encapsulation, are stable, and demonstrate excellent efficiency and tolerability in vivo. The delivery vehicle complexes, therefore, are useful as delivery vehicles for the transportation of the polyanionic cargo encapsulated therein to a target cell. Additionally or alternatively, the delivery vehicle compositions can include a non-anionic cargo. Accordingly, another aspect of the disclosure relates to a delivery vehicle complex comprising: (1) a delivery vehicle composition, as previously described herein, and (2) a polyanionic compound (or cargo). In some implementations, the delivery vehicle composition complexes with one polyanionic compound (e.g., one RNA). In various implementations, the delivery vehicle composition complexes with two different polyanionic compound (e.g., two different RNAs or an RNA and a DNA). In some implementations, the delivery vehicle composition complexes with three or more different polyanionic compounds (e.g., 3, 4, or 5 different RNAs). In some cases, the multicomponent delivery vehicle system complexes with one or more of a nucleic acid selected from DNA and RNA (e.g., an antigenic RNA and adjuvanting DNA, such as CpG).

The delivery vehicle complexes described herein may be characterized by the relative mass ratio of one of the components of the delivery vehicle composition to the cargo (e.g., a polyanionic compound) in the complex. Mass ratios of the components in the delivery vehicle complex can be readily calculated based upon the known concentrations and volumes of stock solutions of each component used in preparing the complex.

Moreover, if non-anionic cargoes are present in the delivery vehicle complex, mass ratios may provide a more accurate representation of the relative amounts of delivery vehicle components to the overall cargo than cation:anion charge ratios, which do not account for non-anionic material. Specifically, the mass ratio of a component refers to the ratio of the mass of this particular component in the system to the mass of the “cargo” in the system. “Cargo” may refer to the total polyanionic compound(s) present in the system. In one example, the polyanionic compound(s) may refer to nucleic acid(s). In one example, the polyanionic compound(s) refer to mRNA(s) encoding at least one protein.

### Post-Formulation

Once ANPs are formed during the formulation process of block (340), several post-processing operations may be completed on the formulation version of process chip (111), as shown in block (350) of FIG. 4. In some versions, these additional processes may include dialysis for buffer exchange and ethanol removal, followed by evaporative concentration to reduce volume for dosing. Other suitable processing steps may be used. Ultimately, the process may yield a ready-to-use therapeutic polynucleotide composition, as shown in block (360). Such therapeutic compositions may include, but are not limited to, cell therapies, oncological treatments, protein replacement, vaccines, expression of effector proteins, inducement of loss of function through expression of dominant negative proteins, and gene/genome editing. Such therapeutic compositions may be delivered to patients in any suitable fashion.

The various sub-processes referred to in FIG. 4 may be carried out using any suitable number or type(s) of process chip (111). In some versions, the entire process shown in FIG. 4 is carried out using a single version of process chip (111). In some other versions, certain sub-processes are carried out on a dedicated process chip (111), while other sub-processes are carried out on another dedicated process chip (111). For instance, in some versions, template preparation (block 310) is carried out on a dedicated template version of process chip (111); IVT transcription and purification (blocks 320, 330) are carried out on a dedicated IVT version of process chip (111); and formulation (block 340) is carried out on a dedicated formulation version of process chip (111).

In versions where certain sub-processes are carried out on a dedicated process chip (111) while other sub-processes are carried out on another dedicated process chip (111), the same instrument of system (100) may be used with the various process chips (111). In some such versions, the same instrument of system (100) accommodates all the process chips (111) that are needed to carry out the process shown in FIG. 4, such that the instrument of system (100) transfers fluids from one process chip (111) to another process chip (111) at the appropriate stage of the process. In some other versions, an instrument of system (100) only accommodates one single process chip (111) at a time. In some such versions, a portion of the process of FIG. 4 (e.g., template preparation (block (310)) may be carried out using a dedicated process chip (111), with the resulting fluid(s) being stored in one or more vials in reagent storage frame (107). That dedicated process chip (111) may then be removed from the instrument of system (100) and be replaced with another dedicated process chip (e.g., a version of process chip (111) dedicated to performing IVT transcription (block (320))), with that second dedicated process chip receiving fluid from one or more vials in reagent storage frame (107) and/or other sources. Different dedicated process chips (111) may thus be used in an appropriate sequence within the instrument of system (100) to carry out the process of FIG. 4.

### Polyanionic Compounds

The delivery vehicle complexes of the disclosure can comprise one or more polyanionic compounds (polyanionic cargo) that can be delivered by the complex to a target in vivo, such as a cell. The polyanionic compound can be complexed to the cationic component (e.g., a compound of Formula (I), such as Compound 140) of the delivery vehicle complex via electrostatic interactions.

In some implementations, the polyanionic compound comprises a nucleic acid. Nucleic acids, as used herein, include naturally occurring nucleic acids (e.g., DNA, RNA, and/or hybrids thereof), as well as unnaturally occurring nucleic acids. Non-limiting examples of unnatural amino acids are those that comprise an unnatural backbone, modified backbone linkages such as phosphorothioate, unnatural or modified bases, and/or unnatural and modified termini. Exemplary nucleic acids include genomic DNA, complementary DNA (cDNA), messenger RNA (mRNA), micro RNA (miRNA), small interfering RNA (siRNA), small activating RNA (saRNA), peptide nucleic acids (PNA), antisense oligonucleotides, ribozymes, plasmids, and immune stimulating nucleic acids.

In some implementations, the polyanionic compound comprises RNA. The RNA may be selected from the group consisting of chemically modified or unmodified RNA, single-stranded or double-stranded RNA, coding or non-coding RNA, mRNA, oligoribonucleotide, viral RNA, retroviral RNA, self-replicating (replicon) RNA (srRNA), tRNA, rRNA, immunostimulatory RNA, microRNA, siRNA, small nuclear RNA (snRNA), small-hairpin (sh) RNA riboswitch, RNA aptamer, RNA decoy, antisense RNA, a ribozyme, or any combination thereof. In some implementations, the nucleic acid cargo is RNA including but not limited to modified mRNAs, self-amplifying RNAs, and circular RNAs. In some implementations, the RNA comprises a coding RNA.

RNA is the usual abbreviation for ribonucleic acid. It is a nucleic acid molecule, i.e. a polymer consisting of nucleotide monomers. These nucleotides are usually adenosine monophosphate (AMP), uridine monophosphate (UMP), guanosine monophosphate (GMP) and cytidine monophosphate (CMP) monomers or analogues thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. Usually RNA may be obtainable by transcription of a DNA sequence, e.g., inside a cell. In eukaryotic cells, transcription is typically performed inside the nucleus or the mitochondria. In vivo, transcription of DNA usually results in the so-called premature RNA (also called pre-mRNA, precursor mRNA or heterogeneous nuclear RNA) which has to be processed into so-called messenger RNA, usually abbreviated as mRNA. Processing of the premature RNA, e.g. in eukaryotic organisms, comprises a variety of different posttranscriptional modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, an open reading frame, optionally a 3′UTR and a poly(A) tail.

In addition to messenger RNA, several non-coding types of RNA exist which may be involved in regulation of transcription and/or translation, and immunostimulation. Within the present disclosure the term “RNA” further encompasses any type of single stranded (ssRNA) or double stranded RNA (dsRNA) molecule known in the art, such as viral RNA, retroviral RNA and replicon RNA, small interfering RNA (siRNA), antisense RNA (asRNA), circular RNA (circRNA), ribozymes, aptamers, riboswitches, immunostimulating/immunostimulatory RNA, transfer RNA (tRNA), ribosomal RNA (rRNA), small nuclear RNA (snRNA), small nucleolar RNA (snoRNA), microRNA (miRNA), and Piwi-interacting RNA (piRNA).

5′-CAP-Structure: A 5′-CAP is typically a modified nucleotide (CAP analogue), particularly a guanine nucleotide, added to the 5′ end of an mRNA molecule. In certain implementations, the 5′-CAP is added using a 5′-5′-triphosphate linkage (also named m7GpppN). Further examples of 5′-CAP structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-CAP structures may be used in the context of the present disclosure to modify the RNA sequence of the present disclosure. Further modified 5′-CAP structures which may be used in the context of the present disclosure are CAP1 (additional methylation of the ribose of the adjacent nucleotide of m7GpppN), CAP2 (additional methylation of the ribose of the 2^{nd} nucleotide downstream of the m7GpppN), CAP3 (additional methylation of the ribose of the 3^{rd} nucleotide downstream of the m7GpppN), CAP4 (additional methylation of the ribose of the 4^{th} nucleotide downstream of the m7GpppN), ARCA (anti-reverse CAP analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In the context of the present disclosure, a 5′ cap structure may also be formed in chemical RNA synthesis or RNA in vitro transcription (co-transcriptional capping) using cap analogues, or a cap structure may be formed in vitro using capping enzymes (e.g., commercially available capping kits).

A cap analogue refers to a non-polymerizable di-nucleotide that has cap functionality in that it facilitates translation or localization, and/or prevents degradation of the RNA molecule when incorporated at the 5′ end of the RNA molecule. Non-polymerizable means that the cap analogue will be incorporated only at the 5′terminus because it does not have a 5′ triphosphate and therefore cannot be extended in the 3′ direction by a template-dependent RNA polymerase.

Cap analogues include, but are not limited to, a chemical structure selected from the group consisting of m7GpppG, m7GpppA, m7GpppC; unmethylated cap analogues (e.g., GpppG); dimethylated cap analogue (e.g., m2,7GpppG), trimethylated cap analogue (e.g., m2,2,7GpppG), dimethylated symmetrical cap analogues (e.g., m7Gpppm7G), or anti reverse cap analogues (e.g., ARCA; m7,2′OmeGpppG, m7,2′dGpppG, m7,3′OmeGpppG, m7,3′dGpppG and their tetraphosphate derivatives) The synthesis of N7-(4-chlorophenoxyethyl) substituted dinucleotide cap analogues has been described recently.

A poly(A) tail also called “3′-poly(A) tail” or “Poly(A) sequence” is typically a long homopolymeric sequence of adenosine nucleotides of up to about 400 adenosine nucleotides, e.g. from about 25 to about 400, from about 50 to about 400, from about 50 to about 300, from about 50 to about 250, or from about 60 to about 250 adenosine nucleotides, added to the 3′ end of an mRNA. In certain implementations of the present disclosure, the poly(A) tail of an mRNA or srRNA is derived from a DNA template by RNA in vitro transcription. Alternatively, the poly(A) sequence may also be obtained in vitro by common methods of chemical synthesis without being necessarily transcribed from a DNA-progenitor. Moreover, poly(A) sequences, or poly(A) tails may be generated by enzymatic polyadenylation of the RNA.

A stabilized nucleic acid, typically, exhibits a modification increasing resistance to in vivo degradation (e.g. degradation by an exo- or endo-nuclease) and/or ex vivo degradation (e.g. by the manufacturing process prior to composition administration, e.g. in the course of the preparation of the composition to be administered). Stabilization of RNA can, e.g., be achieved by providing a 5′-CAP-Structure, a poly(A) tail, or any other UTR-modification. Stabilization can also be achieved by backbone-modification (e.g., use of synthetic backbones such as phosphorothioate) or modification of the G/C-content or the C-content of the nucleic acid. Various other methods are known in the art and conceivable in the context of the disclosure, to stabilize or otherwise improve the function of the nucleic acid. Provided herein, therefore, are polynucleotides which have been designed to improve one or more of the stability and/or clearance in tissues, receptor uptake and/or kinetics, cellular access, engagement with translational machinery, RNA half-life, translation efficiency, immune evasion, immune induction (for vaccines), protein production capacity, secretion efficiency (when applicable), accessibility to circulation, protein half-life and/or modulation of a cell's status, function and/or activity.

A 5′-UTR is typically understood to be a particular section of RNA. It is located 5′ of the open reading frame of the mRNA. In the case of srRNA, the open reading frame encodes the viral non-structural proteins while the sequence of interest is encoded in the subgenomic fragment of the viral RNA. Thus, the 5′UTR is upstream of nsP1 open reading frame. In addition, the subgenomic RNA of the srRNA has a 5′UTR. Thus, the subgenomic RNA containing a sequence of interest encoding a protein of interest contains a 5′UTR. Typically, the 5′-UTR starts with the transcriptional start site and ends one nucleotide before the start codon of the open reading frame. The 5′-UTR may comprise elements for controlling gene expression, also called regulatory elements. Such regulatory elements may be, for example, ribosomal binding sites or a 5′-Terminal Oligopyrimidine Tract. The 5′-UTR may be posttranscriptionally modified, for example by addition of a 5′-CAP. In the context of the present disclosure, a 5′UTR corresponds to the sequence of a mature mRNA or srRNA which is located between the 5′-CAP and the start codon. In one implementations, the 5′-UTR corresponds to the sequence which extends from a nucleotide located 3′ to the 5′-CAP, and in certain implementations from the nucleotide located immediately 3′ to the 5′-CAP, to a nucleotide located 5′ to the start codon of the protein coding region and in some cases to the nucleotide located immediately 5′ to the start codon of the protein coding region. The nucleotide located immediately 3′ to the 5′-CAP of a mature mRNA or srRNA typically corresponds to the transcriptional start site. The term “corresponds to” means that the 5′-UTR sequence may be an RNA sequence, such as in the mRNA sequence used for defining the 5′-UTR sequence, or a DNA sequence which corresponds to such RNA sequence. In the context of the present disclosure, the term “a 5′-UTR of a gene”, such as “a 5′-UTR of a NYESO1 gene”, is the sequence which corresponds to the 5′-UTR of the mature mRNA derived from this gene, i.e. the mRNA obtained by transcription of the gene and maturation of the pre-mature mRNA. The term “5′-UTR of a gene” encompasses the DNA sequence and the RNA sequence of the 5′-UTR.

Generally, the term “3′-UTR” refers to a part of the nucleic acid molecule which is located 3′ (i.e. “downstream”) of an open reading frame and which is not translated into protein. Typically, a 3′-UTR is the part of an RNA which is located between the protein coding region (open reading frame (ORF) or coding sequence (CDS)) and the poly(A) sequence of the mRNA. In the context of the present disclosure, the term 3′-UTR may also comprise elements, which are not encoded in the template, from which an RNA is transcribed, but which are added after transcription during maturation, e.g. a poly(A) sequence. A 3′-UTR of the RNA is not translated into an amino acid sequence.

With respect to srRNA, the 3′-UTR sequence is generally encoded by the viral genomic RNA, which is transcribed into the respective mRNA during the gene expression process. The genomic sequence is first transcribed into pre-mature mRNA. The pre-mature mRNA is then further processed into mature mRNA in a maturation process. This maturation process comprises 5′capping. In the context of the present disclosure, a 3′-UTR corresponds to the sequence of a mature mRNA or srRNA (and the srRNA subgenomic RNA), which is located between the stop codon of the protein coding region, preferably immediately 3′ to the stop codon of the protein coding region for the sequence of interest, and the poly(A) sequence of the mRNA. The term “corresponds to” means that the 3′-UTR sequence may be an RNA sequence, such as in the mRNA sequence used for defining the 3′-UTR sequence, or a DNA sequence, which corresponds to such RNA sequence. In the context of the present disclosure, the term “a 3′-UTR of a gene”, is the sequence, which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence (both sense and antisense strand and both mature and immature) of the 3′-UTR.

According to certain implementations of the disclosure, the RNAs for use in the delivery vehicle complexes herein comprise an RNA comprising at least one region encoding a peptide (e.g., a polypeptide), or protein, or functional fragment of the foregoing. As used herein, “functional fragment” refers to a fragment of a peptide, (e.g., a polypeptide), or protein that retains the ability to induce an immune response. In one implementations, the coding RNA is selected from the group consisting of mRNA, viral RNA, retroviral RNA, and self-replicating RNA. In some implementations, the RNA encodes a viral peptide (e.g., a viral polypeptide), a viral protein, or functional fragment of the foregoing. In various cases, the RNA encodes for a human papillomavirus (HPV) protein or a functional fragment thereof. In some cases, the RNA encodes for a HPV E6 protein, a HPV E7 protein, a combination thereof, or a functional fragment of any of the foregoing. In some cases, the RNA encodes for a viral spike protein or a functional fragment thereof. In various implementations, the RNA encodes for a SARS-CoV spike (S) protein, or a functional fragment thereof. In some cases, the RNA encodes for an influenza protein. In various implementations, the RNA encodes for influenza hemagglutinin (HA), or a functional fragment thereof. In some implementations, the RNA encodes for a combination of the foregoing.

Contemplated viruses for which the RNA of the delivery vehicle complex can encode, include, but are not limited to: Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, humanherpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simplex, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as betacoronavirus, such as SARS-CoV, SARS-CoV-2, MERS-CoV, HCoV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simplex, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever Sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SVV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHoV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

In some implementations, the RNA encodes for adenovirus, alphavirus, calicivirus (e.g., a calicivirus capsid antigen), coronavirus polypeptides, distemper virus, Ebola virus polypeptides, enterovirus, flavivirus, hepatitis virus (AE), herpesvirus, infectious peritonitis virus, leukemia virus, Marburg virus, orthomyxovirus, papilloma virus, parainfluenza virus, paramyxovirus, parvovirus, pestivirus, picorna virus (e.g., a poliovirus), pox virus (e.g., a vaccinia virus), rabies virus, reovirus, retrovirus, and rotavirus. In certain implementations, the RNA encodes for SARS-CoV-2, HPV (e.g., E6 and/or E7), or influenza (e.g., influenza hemagglutinin (HA).

In some implementations, the combined delivery of two or more particular nucleic acids together may be especially useful for therapeutic applications. For example, in some implementations, the one or more polyanionic cargo compounds includes a combination of sgRNA (single guide RNA) as a CRISPR sequence and mRNA encoding Cas9. In still further implementations, the nucleic acids may also be complexed with proteins such as with the CRISPR/Cas9 ribonucleoprotein complex. In some cases, the multicomponent delivery vehicle system complexes with one or more of a nucleic acid selected from DNA and RNA (e.g., an antigenic RNA and adjuvanting DNA, such as CpG).

### Polynucleotide Synthesis

Methods of making polynucleotides of a predetermined sequence are well-known.

Solid-phase synthesis methods are known for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well.

Any method known in the art for making RNA is contemplated herein for making the RNAs. Illustrative methods for making RNA include but are not limited to, chemical synthesis and in vitro transcription.

In certain implementations, the RNA for use in the methods herein is chemically synthesized. Chemical synthesis of relatively short fragments of oligonucleotides with defined chemical structure provides a rapid and inexpensive access to custom-made oligonucleotides of any desired sequence. Whereas enzymes synthesize DNA and RNA only in the 5′ to 3′ direction, chemical oligonucleotide synthesis does not have this limitation, although it is most often carried out in the opposite, i.e. the 3′ to 5′ direction. In certain implementations, the process is implemented as solid-phase synthesis using the phosphoramidite method and phosphoramidite building blocks derived from protected nucleosides (A, C, G, and U), or chemically modified nucleosides.

In some implementations, modifications are included in the modified nucleic acid or in one or more individual nucleoside or nucleotide. For example, modifications to a nucleoside may include one or more modifications to the nucleobase, the sugar, and/or the internucleoside linkage. In some implementations having at least one modification, the polynucleotide includes a backbone moiety containing the nucleobase, sugar, and internucleoside linkage of: pseudouridine-alpha-thio-MP, 1-methyl-pseudouridine-alpha-thio-MP, 1-ethyl-pseudouridine-MP, 1-propyl-pseudouridine-MP, 1-(2,2,2-trifluoroethyl)-pseudouridine-MP, 2-amino-adenine-MP, xanthosine-MP, 5-bromo-cytidine-MP, 5-aminoallyl-cytidine-MP, or 2-aminopurine-riboside-MP.

In other implementations having at least one modification, the polynucleotide includes a backbone moiety containing the nucleobase, sugar, and internucleoside linkage of: pseudouridine-alpha-thio-MP, 1-methyl-pseudouridine-alpha-thio-MP, or 5-bromo-cytidine-MP. Nucleoside and nucleotide modifications contemplated for use in the present disclosure are known in the art.

To obtain the desired oligonucleotide, the building blocks are sequentially coupled to the growing oligonucleotide chain on a solid phase in the order required by the sequence of the product in a fully automated process. Upon the completion of the chain assembly, the product is released from the solid phase to the solution, deprotected, and collected. The occurrence of side reactions sets practical limits for the length of synthetic oligonucleotides (up to about 200 nucleotide residues), because the number of errors increases with the length of the oligonucleotide being synthesized. Products are often isolated by HPLC to obtain the desired oligonucleotides in high purity.

In certain implementations, RNA is made using in vitro transcription. The terms “RNA in vitro transcription” or “in vitro transcription” relate to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which in certain implementations is a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In one implementations of the present disclosure, the DNA template is linearized with a suitable restriction enzyme, before it is transcribed in vitro. The cDNA may be obtained by reverse transcription of mRNA or chemical synthesis. Moreover, the DNA template for in vitro RNA synthesis may also be obtained by gene synthesis.

Methods for in vitro transcription are known in the art. Reagents used in the methods typically include: 1) a linearized DNA template with a promoter sequence that has a high binding affinity for its respective RNA polymerase such as bacteriophage-encoded RNA polymerases; 2) ribonucleoside triphosphates (NTPs) for the four bases (adenine, cytosine, guanine and uracil); 3) in some cases, a cap analogue as defined above (e.g. m7G(5′)ppp(5′)G (m7G)); 4) a DNA-dependent RNA polymerase capable of binding to the promoter sequence within the linearized DNA template (e.g. T7, T3 or SP6 RNA polymerase); 5) optionally a ribonuclease (RNase) inhibitor to inactivate any contaminating RNase; 6) optionally a pyrophosphatase to degrade pyrophosphate, which may inhibit transcription; 7) MgCl2, which supplies Mg2+ ions as a co-factor for the polymerase; 8) a buffer to maintain a suitable pH value, which can also contain antioxidants (e.g. DTT), and/or polyamines such as spermidine at optimal concentrations.

### Pharmaceutical Formulations and Modes of Administration

Also provided herein are pharmaceutical compositions that include the delivery vehicle complexes of the disclosure, and an effective amount of one or more pharmaceutically acceptable excipients. An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females. As used herein, the term “excipient” means any pharmaceutically acceptable additive, carrier, diluent, adjuvant, or other ingredient, other than the active pharmaceutical ingredient (API), suitably selected with respect to the intended form of administration, and consistent with conventional pharmaceutical practices.

The complexes of the disclosure can be administered to a subject or patient in a therapeutically effective amount. The complexes can be administered alone or as part of a pharmaceutically acceptable composition or formulation. In addition, the complexes can be administered all at once, as for example, by a bolus injection, multiple times, or delivered substantially uniformly over a period of time. It is also noted that the dose of the compound can be varied over time.

The delivery vehicle complexes disclosed herein and other pharmaceutically active compounds, if desired, can be administered to a subject or patient by any suitable route, e.g. orally, rectally, parenterally, (for example, intravenously, intramuscularly, or subcutaneously) intracisternally, intravaginally, intraperitoneally, intravesically, or as a buccal, inhalation, or nasal spray. The administration can be to provide a systemic effect (e.g. enteral or parenteral). All methods that can be used by those skilled in the art to administer a pharmaceutically active agent are contemplated.

Compositions suitable for parenteral injection may comprise physiologically acceptable sterile aqueous or non-aqueous solutions, dispersions, suspensions, or emulsions, and sterile powders for reconstitution into sterile injectable solutions or dispersions. Examples of suitable aqueous and non-aqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

When the composition of the disclosure are used as vaccines, it may comprise one or more immunologic adjuvants. As used herein, the term “immunologic adjuvant” refers to a compound or a mixture of compounds that acts to accelerate, prolong, enhance or modify immune responses when used in conjugation with an immunogen (e.g., neoantigens). Adjuvant may be non-immunogenic when administered to a host alone, but that augments the host's immune response to another antigen when administered conjointly with that antigen. Specifically, the terms “adjuvant” and “immunologic adjuvant” are used interchangeably in the present disclosure. Adjuvant-mediated enhancement and/or extension of the duration of the immune response can be assessed by any method known in the art including without limitation one or more of the following: (i) an increase in the number of antibodies produced in response to immunization with the adjuvant/antigen combination versus those produced in response to immunization with the antigen alone; (ii) an increase in the number of T cells recognizing the antigen or the adjuvant; and (iii) an increase in the level of one or more cytokines. Adjuvants may be aluminum based adjuvants including but not limiting to aluminum hydroxide and aluminum phosphate; saponins such as steroid saponins and triterpenoid saponins; bacterial flagellin and some cytokines such as GM-CSF. Adjuvants selection may depend on antigens, vaccines and routes of administrations.

In some implementations, adjuvants improve the adaptive immune response to a vaccine antigen by modulating innate immunity or facilitating transport and presentation.

Adjuvants act directly or indirectly on antigen presenting cells (APCs) including dendritic cells (DCs). Adjuvants may be ligands for toll-like receptors (TLRs) and can directly affect DCs to alter the strength, potency, speed, duration, bias, breadth, and scope of adaptive immunity. In other instances, adjuvants may signal via proinflammatory pathways and promote immune cell infiltration, antigen presentation, and effector cell maturation. This class of adjuvants includes mineral salts, oil emulsions, nanoparticles, and polyelectrolytes and comprises colloids and molecular assemblies exhibiting complex, heterogeneous structures. In one example, the composition further comprises pidotimod as an adjuvant. In another example, the composition further comprises CpG as an adjuvant.

The compounds of the disclosure can be administered to a subject or patient at dosage levels in the range of about 0.1 mg to about 3,000 mg per day. For a normal adult human having a body weight of about 70 kg, a dosage in the range of about 0.01 mg to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

### Methods of Use

The delivery vehicle complexes disclosed herein can be used to deliver the polyanionic compound of the complex (or cargo) to a cell. Accordingly, disclosed herein are methods of delivering a polyanionic compound, such as a nucleic acid (e.g., RNA) to a cell comprising contacting the cell with the delivery vehicle complex or pharmaceutical composition disclosed herein. In some implementations, the cell can be contacted in vitro. In some implementations wherein the cell is contacted in vitro, the cell is a HeLa cell. In other implementations wherein the cell is contacted in vivo, the multicomponent delivery system of the present disclosure is administered to a mammalian subject. A mammalian subject may include but is not limited to a human or a mouse subject. In yet other implementations wherein the cell is contacted ex vivo, the cell is obtained from a human or mouse subject. In some cases, the cell is a tumor cell. In some cases, the cell is a muscle cell.

In some implementations, the one or more polyanionic cargo compounds may be delivered for therapeutic uses. Non-limiting therapeutic uses include cancer, infectious diseases, autoimmune disorders, and neurological disorders. In certain implementations, the complex comprising the multicomponent delivery system and the polyanionic cargo compound is used as a vaccine. Genetic vaccination, or the administration of nucleic acid molecules (e.g., RNA) to a patient and subsequent transcription and/or translation of the encoded genetic information, is useful in the treatment and/or the prevention of inherited genetic diseases but also autoimmune diseases, infectious diseases, cancerous or tumor-related diseases as well as inflammatory diseases. Genetic vaccination is useful for treating or preventing coronavirus. The vaccine target of the majority of these entities is the coronavirus' spike (S) protein, a heavily glycosylated trimeric class I fusion protein that coats the outside of the virus and is responsible for host cell entry. The S protein of SARS-CoV-2 shares high structural homology with SARS-CoV-1 and contains several subunits vital for viral entry into host cells through the angiotensin converting enzyme 2 (ACE2) receptor, including the S1 domain, the S2 domain, and the receptor binding domain (RBD). Thus, the S protein and its subunits, as well as accessible peptide sequences within these domains, are attractive vaccine antigen targets. Further, genetic vaccination is particularly use in the treatment of cancer because cancer cells express antigens, tumors are generally not readily recognized and eliminated by the host, as evidenced by the development of disease

Vaccines. The delivery vehicle complexes of the disclosure are also useful as vaccines, in which the polyanionic compound is an RNA that may encode an immunogen, antigen or neoantigen. The immune system of a host provides the means for quickly and specifically mounting a protective response to pathogenic microorganisms and also for contributing to rejection of malignant tumors. Immune responses have been generally described as including humoral responses, in which antibodies specific for antigens are produced by differentiated B lymphocytes, and cell mediated responses, in which various types of T lymphocytes eliminate antigens by a variety of mechanisms. For example, CD4 (also called CD4+) helper T cells that are capable of recognizing specific antigens may respond by releasing soluble mediators such as cytokines to recruit additional cells of the immune system to participate in an immune response. CD8 (also called CD8+) cytotoxic T cells are also capable of recognizing specific antigens and may bind to and destroy or damage an antigen-bearing cell or particle. In particular, cell mediated immune responses that include a cytotoxic T lymphocyte (CTL) response can be important for elimination of tumor cells and cells infected by a microorganism, such as virus, bacteria, or parasite. The delivery vehicle complexes of the disclosure have been found to induce immune responses when one or more of the polyanionic compound of the complex encodes a viral peptide (e.g. a viral polypeptide), a viral protein, or functional fragment of the foregoing. For example, delivery vehicle complexes comprising either DV-140-F2 or DV-140-F6 complexed with mRNA encoding the HPV E6/E7 oncogene, a construct of the SARS-CoV spike (S) protein, and/or influenza hemagglutinin (HA) elicited strong humoral and cellular immune responses.

Thus, the disclosure includes methods for inducing an immune response in a subject in need thereof, comprising administering to the subject an effective amount of the delivery vehicle complex (e.g., formulated as an antigenic composition) of the disclosure. Also disclosed herein is a method of treating a viral infection in a subject in need thereof, comprising administering to the subject an effective amount of the delivery vehicle complex of the disclosure. In some implementations, the administering is by intramuscular, intratumoral, intravenous, intraperitoneal, or subcutaneous delivery.

In various implementations, administering the delivery vehicle complexes of the disclosure (e.g., formulated as a composition, pharmaceutical formulation, or antigenic composition) to a subject can result in an increase in the amount of antibodies (e.g., neutralizing antibodies) against the viral antigen that is produced in the subject relative to the amount of antibodies that is produced in a subject who was not administered the delivery vehicle complex. In some implementations, the increase is a 2-fold increase, a 5-fold increase, a 10-fold increase, a 50-fold increase, a 100-fold increase, a 200-fold increase, a 500-fold increase, a 700-fold increase, or a 1000-fold increase.

The immune response raised by the methods of the present disclosure generally includes an antibody response, preferably a neutralizing antibody response, maturation and memory of T and B cells, antibody dependent cell-mediated cytotoxicity (ADCC), antibody cell-mediated phagocytosis (ADCP), complement dependent cytotoxicity (CDC), and T cell-mediated response such as CD4+, CD8+. The immune response generated by the delivery vehicle complexes comprising RNA that encodes a viral antigen as disclosed herein generates an immune response that recognizes, and preferably ameliorates and/or neutralizes, a viral infection as described herein. Methods for assessing antibody responses after administration of an antigenic composition (immunization or vaccination) are known in the art and/or described herein. In some implementations, the immune response comprises a T cell-mediated response (e.g., peptide-specific response such as a proliferative response or a cytokine response). In some implementations, the immune response comprises both a B cell and a T cell response. Antigenic compositions can be administered in a number of suitable ways, such as intramuscular injection, intratumoral injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

Cancer. Various cancers (e.g., cervical cancer) may be treated with the polyanionic cargo compounds delivered by the delivery vehicle complexes of the present disclosure. As shown in Example 8, DV-140-F2 complexed to an mRNA encoding for HPV E6/E7 eliciting both strong cellular and humoral immune responses, illustrating the ability of the delivery vehicle complexes of the disclosure to treat cancer. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

As a non-limiting example, the carcinoma which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

In some implementations, the delivery vehicle complexes of the disclosure are used to treat a cancer is selected from the group consisting of cervical cancer, head and neck cancer, B-cell lymphoma, T-cell lymphoma, prostate cancer, and lung cancer. In some implementations, the delivery vehicle complexes can be used to treat cervical cancer.

Infectious Diseases. In some implementations, the delivery vehicle complexes of the present disclosure is used to treat infectious diseases, such as microbial infection, e.g., a viral infection, a bacterial infection, a fungal infection, or a parasitic infection. Non-limiting examples of infectious diseases include hepatitis (such as HBV infection or HCV infection), RSV, influenza, adenovirus, rhinovirus, or other viral infections.

Autoimmune diseases. Various autoimmune diseases and autoimmune-related diseases may be treated with the delivery vehicle complexes of the present disclosure. As used herein, the term “autoimmune disease” refers to a disease in which the body produces antibodies that attack its own tissues. As a non-limiting example, the autoimmune disease may be Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome**, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia**, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosis, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA).

Neurological diseases. Various neurological diseases may be treated with the delivery vehicle systems of the present disclosure. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS-Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia-Semantic, Dementia Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus-Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Klüver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus-Neurological Sequelae, Lyme Disease-Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Post infectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease-Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Shortlasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

In jurisdictions that forbid the patenting of methods that are practiced on the human body, the meaning of “administering” of a composition to a human subject or patient shall be restricted to prescribing a controlled substance that a human subject or patient will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

Example Applications of mRNA Therapeutics

First generation SARS-CoV2 vaccines are produced by immunizing individuals with antigens that encode the Spike protein encoded by the Wuhan SARS-CoV2 virus.

Different vaccine formats are generated in which the antigen is delivered as mRNA nanoparticles, formulated recombinant protein or adenoviruses all of which presented pre-fusion stabilized versions of the native spike protein. Despite the success in swiftly developing effective vaccines against SARS-CoV2, successive waves of infection partly driven by the rapid evolution of the virus have resulted in the rise of new strains (alpha, beta, gamma and delta) that have successively become dominant in the population of infected individuals. These variants have been characterized by higher infection rates and in the case of the delta strain, higher rates of breakthrough infection in vaccinated individuals.

The therapeutics provided may provide the benefit of overcoming the increasingly lower efficacy of first-generation vaccines against rapidly arising viral strains. One approach, as provided by the examples herein, is to employ more distantly related spike proteins originating from the betacoronavirus genus as a booster vaccination strategy to induce a broadly neutralizing response. Specifically, individuals that have been previously infected by SARS-CoV2 or that have been vaccinated by first-generation COVID vaccines will be given booster vaccinations with mRNA-based vaccines encoding spike proteins from related betacoronavirus species that infect humans or other hosts such as, rodents, bats (Rhinolophus genus) and pangolins (Philidota). Not to be bound by any particular theory, but it is believed that by the examples described here, similar to successive vaccinations against more distantly related versions of the same antigen, may drive the adaptive immune system to focus and amplify responses to antigenic determinants that are more deeply conserved among the antigens rather than the more immunodominant epitopes that dominate the immune response against any one given viral strain. In addition, the approach described in the examples herein may allow the identification of specific antibody sequences (re-arranged heavy and light chain B-cell receptors) capable of driving in vivo neutralization of diverse SARS-CoV2 strains. Such identified sequences may be used for the production of recombinant antibody cocktails (either as recombinant protein or mRNA based therapeutics) that can be used in infected patients requiring therapeutic intervention.

The mRNA provided herein may be employed as therapeutic against any of the viral infection provided above. For example, one method herein is related to a method to treat a human patient having antibodies to a first virus, comprising: extracting a sequence of a spike protein of a second virus from at least one first non-human mammal that is previously exposed to an infection by the second virus; identifying a target antigen specific to the spike protein; and injecting an mRNA therapeutic comprising an mRNA encoding the target antigen into the human patient.

The patient may be any suitable individual in need of the therapeutic.

In one implementation, each of the first virus and the second virus is a type of coronavirus, which is a group of viruses already provided above. In one implementation of this aspect, wherein the first virus and the second virus are not the same. In another implementation, these two viruses are the same. For example, in one implementation, the first virus is SARS-COV-2 virus, and the second virus is SARS-COV-1 virus. In another implementation, the first virus is SARS-COV-1 virus, and the second virus is SARS-COV-2 virus. In one implementation, the first virus is a betacoronavirus. In one implementation, the second virus is a betacoronavirus.

In one implementation the first non-human mammal is a bat, a pangolin, a rodent, or any combinations thereof. As described above, other mammals that are known to host related betacoronavirus species, such as rodents, are also possible. Betacoronavirus (β-CoVs or Beta-CoVs) is one of four genera of coronaviruses. Member viruses are enveloped, positive-strand RNA viruses that infect mammals (of which humans are part).

In some instances, the natural reservoir for betacoronaviruses are bats and rodents. Rodents are the reservoir for the subgenus Embecovirus, while bats are the reservoir for the other subgenera. One example of the betacoronaviruses of concerning humans are OC43 and HKU1 (which can cause the common cold) of lineage A, SARS-CoV and SARS-CoV-2 (which causes the disease COVID-19) of lineage B, and MERS-CoV of lineage C. MERS-CoV is the first betacoronavirus belonging to lineage C that is known to infect humans. Any of these betacoronaviruses described herein may be employed as part of the implementations described herein.

In one implementation, the target antigen is a viral antigen specific to the first virus. In one implementation, the mRNA therapeutic comprises the mRNA encapsulated in a delivery vehicle composition. The antigen and delivery vehicle compositions are provided above.

Another example application of the mRA therapeutic described herein is a method to treat a human patient having antibodies to a first virus, comprising: extracting a sequence of a spike protein of a first virus from at least one human subject that has antibodies with respect to the second virus; identifying a target antigen specific to the spike protein; and injecting an mRNA therapeutic comprising an mRNA encoding the target antigen into the human patient.

In one implementation, the human subject and the human patient are not the same person. In another implementation, the subject and the patient are the same person. The patient may be any suitable individual in need of the therapeutic.

The human patient may have acquired certain immunity (E.g., in the form of presence of antibodies) against certain disease. In one implementation where in the disease is caused by infection of a coronavirus, the human patient is 1) previously exposed to an infection by the first coronavirus, 2) subjected to a vaccination regiment against the first coronavirus, or both. In one implementation, each of the first virus and the second virus is a type of coronavirus. In one implementation, the first virus and the second virus are not the same. In one implementation, the first virus is SARS-COV-2 virus, and the second virus is SARS-COV-1 virus. In one implementation of this aspect, the target antigen is a viral antigen specific to the first virus. In one implementation of this aspect, the mRNA therapeutic comprises the mRNA encapsulated in a delivery vehicle composition.

In any of methods provided herein, the method may further comprise identifying an antibody sequence adapted to in vivo neutralization of a plurality of strains of the second virus.

The presently described technology and its advantages will be better understood by reference to the following examples. These examples are provided to describe specific implementations of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### Examples

### Example 1. In Vivo Vaccination Studies

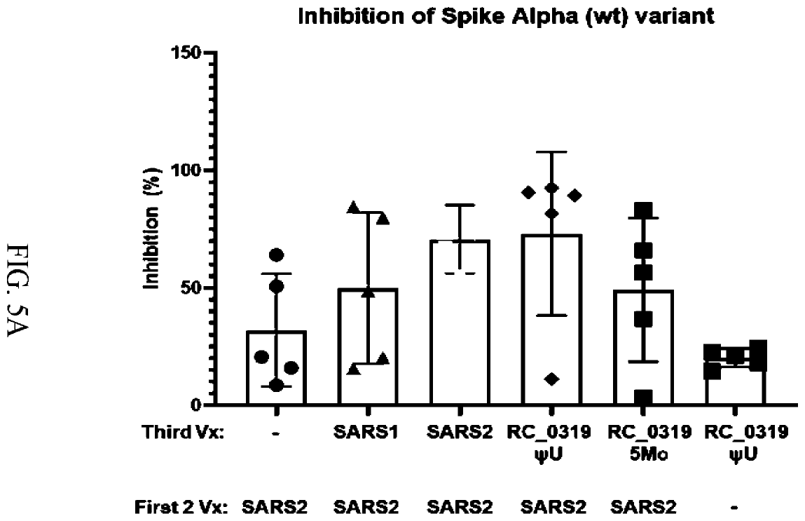

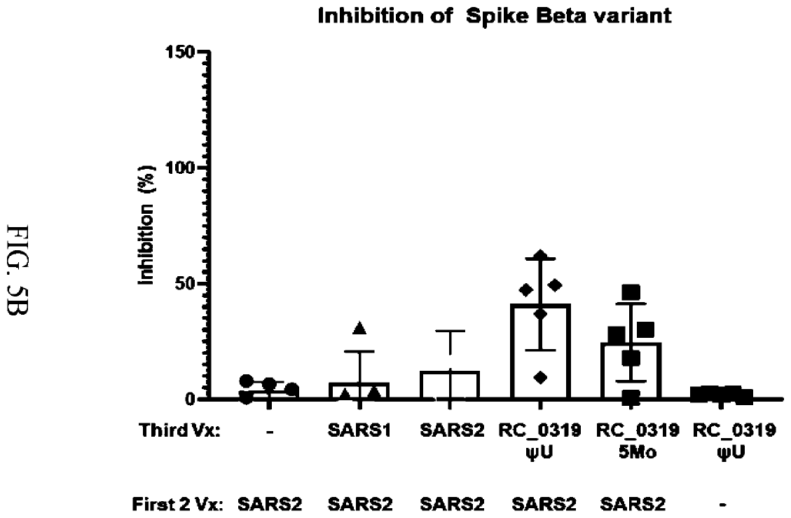

Six to eight-week-old Balb/c mice were vaccinated intramuscularly with two doses given 21 days apart of 1 μg mRNA coding for the WT (alpha) SARS-CoV2 spike protein. On day 42, mice (n=5) were boosted using mRNA coding for the spike protein of the WT SARS-CoV2, SARS-CoV1 or RC_0319. The control groups included five mice that did not receive a boost on day 42 and five mice received only one dose of mRNA coding for the RC-0319 spike on day 42. WT SARS-CoV2 and SARS-CoV1 mRNA were synthetized using N1 methylpseudouridines. RC_0319 mRNA was synthetized using either 5methilpseudouridine or 5 methoxyuridines. On Day 58, the serum and spleen were collected from all animals. As shown in FIG. 5A, the ability of the serum to neutralize the WT SARA-CoV2 and the Beta SARS-CoV2 was assessed using the SARS-CoV-2 Surrogate Virus Neutralization Test (sVNT) Kit from Genscript following manufacturer protocol.

For the Luminex inhibition assay, one million streptavidin beads were conjugated to 5 μg biotinylated WT or Beta SARS-CoV2 Spike protein (Acro biosystems). ACE2-hFC fusion protein (Acro biosystems) was conjugated to PE using the SiteClick™ Antibody Labeling Kits (Invitrogen) per manufacturer instructions. Spike protein conjugated beads were incubated with 10× diluted serum for 1 hour. PE-ACE2-FC was then added to the beads and incubated for an additional hour. The beads were washed using PBS supplemented with 0.05% tween 20 before they were analyzed on a Luminex Magpix. The control groups were the positive and negative control from the Genscript surrogate Virus Neutralization kit and a serum sample from a patient that received 3 doses of FDA approved mRNA COVID19 vaccine. Inhibition was calculated as inhibition=(1−MFI value of Sample/MFI value of Negative Control)×100%.

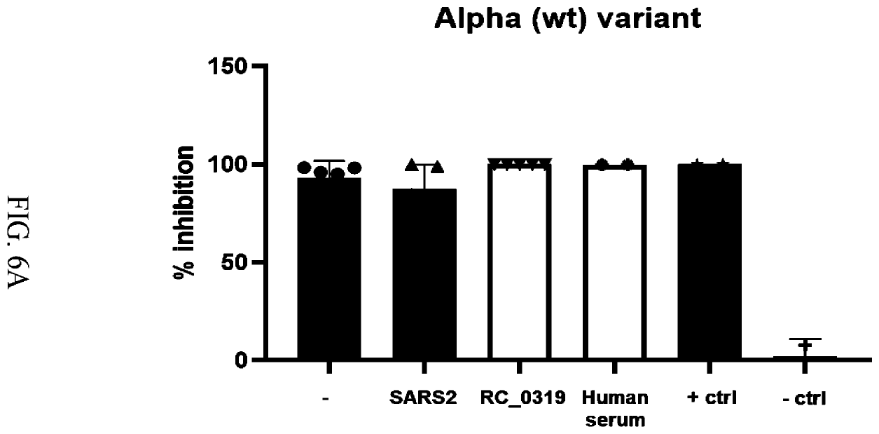

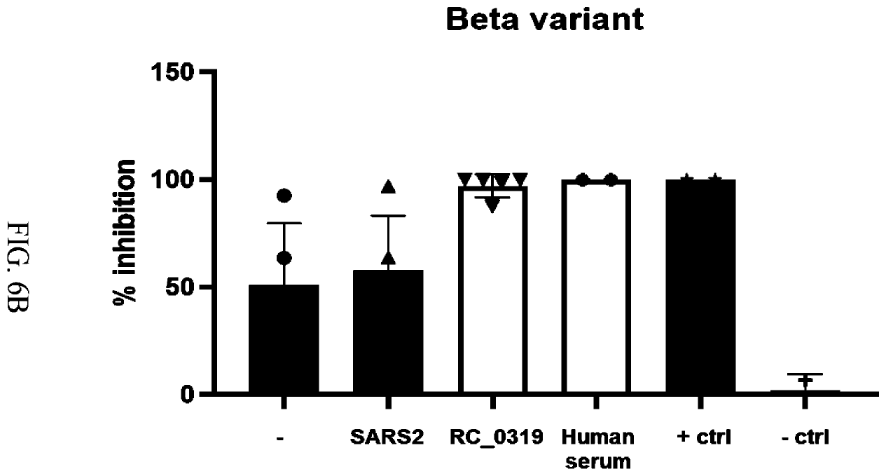

Results: In this study, Balb/c mice (n=5) were dosed twice with SARS2-targeted mRNA-based vaccine three weeks apart to mimic current approved COVID vaccines. As depicted in FIGS. 5A and 5B, animals were boosted three weeks later with mRNA encoding spike from SARS1, SARS2, or RC_0319. Control groups included animals that did not receive a third vaccination and animals that only received a single RC_0319 vaccination. Serum was collected and neutralization was assessed using surrogate neutralization assay (Genscript) with receptor binding domain of either alpha (wildtype) variant (FIG. 5A) or beta (FIG. 5B) variant. Serum was collected and neutralization assessed by inhibition of receptor binding domain from alpha (WT) (FIG. 6A) or beta spike (FIG. 6B) binding to ACE2 was performed by Luminex. RC_0319 provided better neutralization against the spike beta mutant than a 3^{rd} vaccine with the alpha (WT) spike protein. The phylogenetically distinct spike enhanced neutralizing immunity against the SARS2 beta variant.

### Example 2: Flow Neutralization Assay

For the flow neutralization assay, HeLa cells were transfected using mRNA coding for the WT or omicron SARS-CoV2 Spike protein using MessengerMax (Invitrogen) per manufacturer's protocol. The next day, cells were harvested and incubated with 10× diluted serum for 30 min then PE ACE2-FC was added to the cells at 200× dilution and cells were further incubated at RT for 15 min. Cells were then washed using cell staining buffer (biolegend) and analyzed on a Cytek Flow cytometer. Inhibition was calculated as inhibition=(1−MFI value of Sample/MFI value of Naïve Control)×100%.

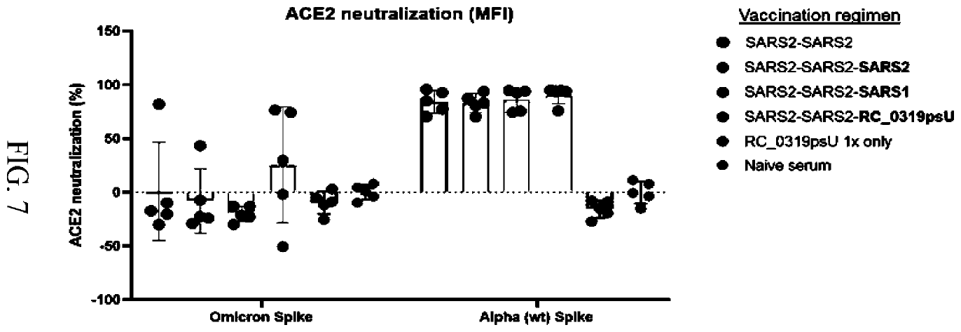

Results: In this study, balb/c mice (n=5) were dosed twice with SARS2-targeted mRNA-based vaccine three weeks apart to mimic current approved COVID vaccines. Animals were boosted three weeks later with mRNA encoding spike from SARS1, SARS2, or RC_0319. Control groups included animals that did not receive a third vaccination and animals that only received a single RC_0319 vaccination. Serum was collected and as shown in FIG. 7, the ability of the serum to inhibit alpha (WT) or omicron spike protein from binding to the ACE2 receptor was assessed by flow cytometry. HeLa cells were used to express alpha or omicron spike proteins. RCO319 boost enhanced the neutralization of the omicron spike binding to the ACE2 receptor, without decreasing the neutralization of the alpha spike. The phylogenetically distinct spike enhanced neutralizing immunity against the SARS2 omicron variant.

### Example 3: T Cells Recall Assay

In the T cells recall assay, one million splenocyte were cultured overnight in RPMI supplemented with 10% FCS, 100 units/mL of penicillin and 100 μg/mL of streptomycin and 55 μM 2-Mercaptoethanol. The next day cells were incubated with 5 μg/ml WT spike RBD protein in the presence of GolgiPlug (BD) for 5 hours at 37C with 5% CO2. Subsequently, the cells were stained with the Zombie NIR live/dead stain (Invitrogen) and then with V500 anti-CD90.2, FITC anti-CD4, BV421 anti-CD8, PE-Cy7 anti-IFNg, APC anti-TNFa and PE anti-IL2. Cytofix/Cytoperm (BD) was used to fix and permeabilize cells. Cells were analyzed on a Cytek Flow cytometer.

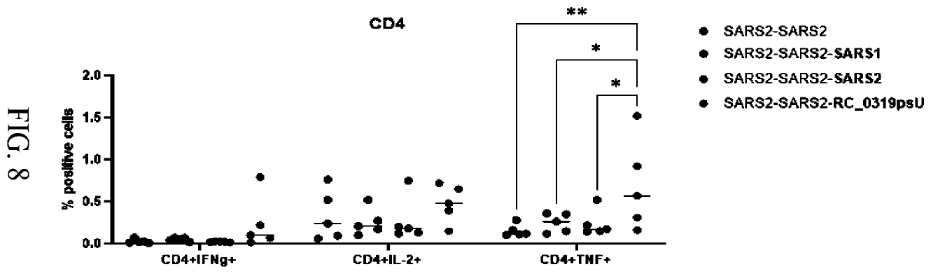

Results: In this study, Balb/c mice (n=5) were dosed twice with SARS2-targeted mRNA-based vaccine three weeks apart to mimic current approved COVID vaccines. Animals were boosted three weeks later with mRNA encoding spike from SARS1, SARS2, or RC_0319. Control groups included animals that did not receive a third vaccination and animals that only received a single RC_0319 vaccination. The spleens that were collected were harvested for two weeks post-second vaccination. The immune responses were assessed by intracellular cytokine staining using alpha (WT) receptor binding domain protein as stimulation. (FIG. 8) The phylogenetically distinct spike enhanced SARS-2 specific T cell immunity.

The sequences are appended to the disclosure as Table 2 in Appendix. The trees and homology tables are provided below.

TABLE 1
Trees and homology Tables

| | | | | | | | | | | | | | | |
|---|---|---|---|---|---|---|---|---|---|---|---|---|---|---|
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 1 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 2 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 3 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 4 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 5 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 6 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 7 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 8 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 9 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 10 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 11 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 12 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 13 | | | | | | | | | | | | | | |
| SEQ ID | [symbol] | [symbol] | [symbol] | [symbol] | [symbol] | [symbol] | [symbol] | [symbol] | [symbol] | [symbol] | [symbol] | [symbol] | [symbol] | [symbol] |
| NO: 14 | | | | | | | | | | | | | | |

[symbol]

indicates data missing or illegible when filed

It should be appreciated that all combinations of the foregoing concepts and implementations and additional concepts and implementations discussed in greater detail below are contemplated as being part of the inventive subject matter disclosed herein, and may be employed in any suitable combination to achieve the benefits as described here. In particular, all combinations of claimed subject matter appearing at the end of this disclosure are contemplated as being part of the inventive subject matter disclosed herein. The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular implementations, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

1. A method to treat a human patient having antibodies to a first virus, comprising: extracting a sequence of a spike protein of a second virus from at least one first non-human mammal that is previously exposed to an infection by the second virus;
identifying a target antigen specific to the spike protein; and
injecting an mRNA therapeutic comprising an mRNA encoding the target antigen into the human patient.

2. The method of claim 1, where each of the first virus and the second virus is a type of coronavirus.

3. (canceled)

4. The method of claim 1, wherein the first virus is SARS-COV-2 virus, and the second virus is SARS-COV-1 virus or wherein the first virus is SARS-COV-1 virus, and the second virus is SARS-COV-2 virus.

5. (canceled)

6. The method of claim 1, wherein the at least one first non-human mammal is a bat, a pangolin, a rodent, or any combinations thereof.

7. The method of claim 1, wherein the target antigen is a phylogenetically distant antigen variant of a spike protein of the first virus.

8. The method of claim 1, wherein the mRNA therapeutic comprises the mRNA encapsulated in a delivery vehicle composition.

9. The method of claim 1, wherein the sequence is selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, or SEQ ID NO:14.

10. The method of claim 1, wherein the second virus is a betacoronavirus.

11. A method to treat a human patient having antibodies to a first virus, comprising: extracting a sequence of a spike protein of a first virus from at least one human subject that has antibodies with respect to a second virus;
identifying a target antigen specific to the spike protein; and
injecting an mRNA therapeutic comprising an mRNA encoding the target antigen into the human patient.

12. The method of claim 11, wherein the human subject and the human patient are not the same person.

13. The method of claim 11, wherein the human patient is (a) previously exposed to an infection by the first virus, (b) previously subjected to a vaccination regiment against the first virus, or (c) both.

14. The method of claim 11, wherein each of the first virus and the second virus is a type of coronavirus.

15. (canceled)

16. The method of claim 11, wherein the first virus is SARS-COV-2 virus, and the second virus is SARS-COV-1 virus.

17. The method of at claim 11, wherein the target antigen is a viral antigen specific to the first virus.

18. The method of a claim 11, wherein the mRNA therapeutic comprises the mRNA encapsulated in a delivery vehicle composition.

19. The method of a claim 11, wherein the second virus is a betacoronavirus.

20. The method of claim 11, wherein the sequence is selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, or SEQ ID NO: 14.

21. The method of claim 1, wherein the target antigen is a phylogenetically distant antigen variant of a spike protein of the first virus.

22. The method of claim 1, further comprising identifying an antibody sequence adapted to in vivo neutralization of a plurality of strains of the second virus.

23. The method of claim 11, wherein the target antigen is a phylogenetically distant antigen variant of a spike protein of the first virus.

24. The method of claim 11, further comprising identifying an antibody sequence adapted to in vivo neutralization of a plurality of strains of the second virus.
